(19) United States
(12) Patent Application Publication
(10) Pub. No.: US 2024/0327643 A1
(43) Pub. Date: Oct. 3, 2024

(54) DNA TEMPLATING OF DYES INTO LONG POLYMER DYE AGGREGATES (SUPERDYES) USING MONOMER OR AGGREGATE SUB-UNITS

(71) Applicant: Boise State University, Boise, ID (US)

(72) Inventors: William B. Knowlton, Boise, ID (US); Bernard Yurke, Boise, ID (US); Olga A. Mass, Boise, ID (US); Keitel Abraham Cervantes Salguero, Boise, ID (US)

(21) Appl. No.: 18/625,767

(22) Filed: Apr. 3, 2024

Related U.S. Application Data
(60) Provisional application No. 63/493,820, filed on Apr. 3, 2023.

(51) Int. Cl. C09B 62/357; G16B 50/30

(52) U.S. Cl. CPC C09B 62/357; G16B 50/30

(57) ABSTRACT

Chemical (e.g., click chemistry), enzymatic- or photo-induced polymerization or other polymerization approaches (e.g., thermally activated) of (1) single dyes (monomer) can be used to produce an extended dye network in which each successive dye is arranged in a head-to-tail arrangement (J-like packing arrangement) of their transition dipole moments or (2) dye aggregate sub-units to achieve polymer branching. Furthermore, various routing patterns are achieved by templating a linear series of dyes onto DNA oligomers of various configurations. Dye aggregate dye sub-unit junctions (e.g., triad, tetrad, pentad, hexad, etc.) are used to achieve polymer branching enable creating various circuit patterns and circuit elements (e.g., optical transistors, gates, etc.). Branched configurations can occur on any surface (e.g., DNA nanostructure, chips substrate, hydrogel, etc.) using DNA (or any similar specific [bio]chemistry).

### CROSS REFERENCE TO RELATED APPLICATIONS

This application claims priority under 35 U.S.C. § 119 to provisional patent application U.S. Ser. No. 63/493,820, filed Apr. 3, 2023. The provisional patent application is herein incorporated by reference in its entirety, including without limitation: the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant Number ECCS 1648655, awarded by the National Science Foundation (INSPIRE); Grant Number N00014-19-1-2615, awarded by the Office of Naval Research; and Grant Number DE-SC0020089, awarded by the Department of Energy. The government has certain rights in the invention.

### TECHNICAL FIELD

The present invention relates generally to quantum computing and/or optical information processing. More particularly, but not exclusively, the present invention relates to DNA templates of dyes into long polymer dye aggregates with non-conjugating bridges (superdyes) using monomer or aggregate sub-units.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

An exciton is the name for the energy packet that resides on a chromophore when it is in its excited state. When two molecules are very close to each other the energy of an excited chromophore can be transferred to a neighboring chromophore without energy loss, in contrast to the usual fluorescence resonance energy transfer (FRET) where energy loss occurs in the transfer. The packet of energy can be exchanged in a wave like manner back and forth between the two molecules. The energy packet, in this sense, acts like a quantum mechanical particle that can become delocalized or spread out over an aggregate of chromophores just like an electron can spread out its wave function over an entire molecule (molecular orbital). The Davydov splitting and the circular dichroism (CD) spectra seen in chromophore aggregates is a manifestation of this delocalization. Davydov splitting is analogous to the splitting of orbitals into bonding and antibonding orbitals when two atoms are brought close together.

Exciton wires can be made by closely spacing chromophores together in a row. As discussed above, when chromophores are nanospaced apart, an exciton may transfer from one chromophore to another without the loss of energy. An exciton created at one end of the row of chromophores may propagate down the row, hopping from one chromophore nanospaced to the next. This is done in a wavelike manner.

Exciton circuits made from these exciton wires may be made to be analogous to electronic circuits but where excitons carry the signals rather than electrons in classical computing. By bringing two exciton wires sufficiently close to each other an exciton can hop from one wire to the other by transferring from one chromophore to another. By doing this carefully one can make devices that function as signal dividers. The division ratio depends on rates with which excitons can be transferred between chromophores in the coupling region. The transfer rate depends on the spacing between the chromophores, their orientations. This dependence on spacing and orientation enables the construction of signal dividers with, for practical purposes, any division ratio. An exciton propagating down one exciton wire will become delocalized so that one must think of the exciton as being in a superposition state where it resides on both exciton wires. This device is a basis-change gate. Its function is analogous to that of an optical beam splitter or microwave directional coupler. A basis-change gate is one of the fundamental quantum gates.

Another quantum gate of fundamental importance is a phase-shift gate. The phase accumulated by an exciton is proportional to the distance it travels. Hence, a phase-shift gate can simply be made by engineering the wire that the exciton travels over to have the length needed to accumulate the proper amount of phase. The phase an exciton accumulates is also determined by its energy relative to the optical transition energy for the chromophore. The optical transition frequency here denotes the energy difference between the chromophore's ground electronic state and its lowest excited electronic state that has an allowed optical transition. Hence, phase shifters can also be fashioned choosing the chromophores of differing optical transition energies. It is also possible to make phase shifters by terminating two ports of a signal divider with chromophores having differing optical transition energies.

Another quantum gate of fundamental importance is a controlled basis change gate. In contrast to the gates already discussed, which rely on wave interference effects, a controlled basis change gate relies on the interaction between two excitons. When two excitons reside on neighboring chromophores they feel each other's presence just like two electrons will feel each other's Coulomb repulsion when they are brought close together. The two exciton interaction arises from static Coulomb interactions between molecules and is most strong when the molecules have an asymmetric molecular structure. Asymmetric molecules possess a permanent electric dipole which changes sign when the molecule is excited from the ground state to the excited state. The static Coulomb interaction, in this case is a dipole-dipole interaction which, when both chromophores are excited (the two exciton case), differs in sign from the case when only one chromophore is excited (the one exciton case). Due to the static Coulomb interactions between chromophores one exciton will accumulate extra phase in the presence of the other exciton. As a result, the presence or absence of one exciton can control how the other exciton moves through a basis change gate.

These three types of gates, the basis-change gate, the phase-shift gate, and the controlled basis-change gate, form a complete set if the phase-shift gates can be produced with a finite set of phase angles. Since the phase angles can be controlled by the exciton path length, the optical transition energies of the phase-shifter wire chromophores or through the construction of optical phase-shifter gates out of basis-change gates with selected ports terminated this last requirement can be met. With this finite set of gates, one can assemble exciton circuits that perform any quantum computation. A set of gates having this property is said to be capable of universal quantum computation. This is analogous to the electronic computer case where the NAND gate is a universal gate in that any Boolean function can be implemented by a circuit employing only NAND gates.

It is possible to perform universal quantum computation with just basis-change gates and phase-shift gates, but the number of parts (gates) one needs grows exponentially with the size of the problem. So, doing quantum computation this way performs as well as classical computers. By introducing basis-change gates one can drastically reduce the parts count so that much fewer parts are required than for a classical computer. The controlled basis-change gate enables the entanglement of many-body (many-exciton) states so that a network of quantum gates acts as if it is performing many different computations simultaneously. This is similar to an n bit memory register. In a classical computer each memory element of the register can be in either a zero or a one state but not both simultaneously. In contrast a quantum mechanical register can be in a state that is a quantum mechanical superposition of being in the zero state and the one state. An n bit quantum memory in this sense can act as if it is holding 2n bits of information whereas the classical computer memory only holds n bits of information. A single controlled basis change gate excepting as inputs the contents of memory elements i and j and delivering the outputs back to memory elements i and j can update the amplitudes of all of the 2n states in the superposition of the memory register simultaneously. This is quantum parallelism.

Not all math problems are known to benefit from quantum speedup, but several classes of problems are known where quantum computers can vastly outperform classical computers. Two of the problems are factoring and database searching.

However, chromophores exhibit many non-ideal characteristics for quantum computing. The electronic degrees of freedom are strongly coupled to the vibrational and environmental degrees of freedom. This causes phase jitter, which causes phase error to grow with time, thus spoiling the interference effects that the quantum gates relay on. All quantum computers suffer from this problem to a greater or lesser extent. Chromophores are also difficult to arrange in the requisite configurations.

The present inventors have previously created compositions of one or more aggregates of two or more chromophores attached to a nucleotide architecture. When two or more chromophores are spaced close enough to form a moderate to large dipole-dipole coupling, they form an aggregate. When aggregates are placed in moderate to weak coupling, an exciton may be transferred from one aggregate to another without energy loss within the nucleotide-templated aggregate architecture, they can propagate excitons down quantum wires and through quantum circuits, which can be used in quantum computing. The use of aggregates in quantum computing has several benefits over traditional methods, including functioning in a noisy environment and being able to be performed at room temperatures. The use of the nucleotide architecture allows for self-assembly of complex structures which place the chromophores and aggregates thereof in precise locations as well as placing the chromophores and aggregates sufficiently close to each other to allow for the transfer of excitons without energy loss, while preventing all of the chromophores to merely form a single aggregate by controlling the amount of coupling between chromophores and aggregates. Furthermore, the use of nucleotides has benefits over the use of proteins for an architecture due to having less complex design rules.

That being said, not only creating, but also controlling the length and routing of, excitonic waveguides with emissive aggregates (i.e., J-aggregates) is a fundamental challenge in the use of molecular quantum materials. Thus, there exists a need in the art to provide a feasible path forward for creating linear, two-, and three-dimensional arrays of emissive aggregates and subsequently controlling their length and routing through DNA templating.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to enable the ability to build and create excitonic circuits and circuit elements including switches, logic gates, wave splitters, interferometers, antennas (light harvesting or otherwise), and quantum logic gates. Exciton quantum computers have fast switching time, a compact size (the components are relatively small molecules), and possible room temperature operation. Since photons are readily converted into excitons and excitons are readily converted into photons, excitonic devices find applications in optical information processing, apart from quantum computing, as more compact embodiments of the currently employed optical phase shifters, signal dividers, and switches, employing Kerr nonlinearities, that have the functionality of controlled basis change gates. For these applications, the performance requirements are less demanding than that for quantum computation.

It is a further object, feature, and/or advantage of the present disclosure to allow emissive aggregates to play key roles in facilitating such applications as detecting, measuring, and controlling quantum entanglement, light harvesting (energy conversion of light to electricity and/or chemical energy), and in developing quantum gates leading to room temperature quantum computing capabilities. The technologies described in co-pending, co-owned Pre-grant Pub. No. 2019/0048036 (U.S. Ser. No. 16/100,052), titled “Excitonic Quantum Computing Mediated by Chromophore-Embedded 1-, 2-, and 3-Dimensional DNA Scaffolds” and U.S. Pre-grant Pub. 2023/0147320 (U.S. Ser. No. 17/249,159), titled “Excitonic Quantum Computing via Aggregate-Aggregate Coupling”, both stand to benefit from the use of DNA templates of dyes into long polymer dye aggregates with non-conjugating bridges (superdyes) using monomer or aggregate sub-units. The disclosures of the aforementioned co-owned, co-pending patent applications are each hereby incorporated by reference herein in their entireties, including without limitation, the specification, claims, and abstract, as well as any figures, tables, appendices, or drawings thereof.

It is still yet a further object, feature, and/or advantage of the present disclosure to develop, test, and/or specify optimal configurations for DNA templates of dyes into long polymer dye aggregates with non-conjugating bridges (superdyes) using monomer or aggregate sub-units. For example, depending on the application, certain types of dyes may are specified, optimal click-chemistry moieties (i.e., dye-end groups) are designed, and/or the synthesis of the dyes with these groups is controlled.

It is still yet a further object, feature, and/or advantage of the present disclosure to address identification of the conditions of dye polymerization on DNA, including a self-limiting process step to control the length of the polymer, and control of how polymer dyes with linkers on each dye will compete for linker sites on DNA.

It is still yet a further object, feature, and/or advantage of the present disclosure to tune the angle between J and K on the dye relative to the transmission coefficient of the exciton in the transmission line.

It is still yet a further object, feature, and/or advantage of the present disclosure to start with certain J and K (magnitude, angle) in the transmission line and then change J and K (magnitude, angle). For example, the change can be a single change, multiple changes, or periodic changes.

DNA templates of dyes into long polymer dye aggregates with non-conjugating bridges (superdyes) using monomer or aggregate sub-units disclosed herein can be used in a wide variety of applications. For example, the excitonic circuits and circuit elements that are built through the use of DNA templates of dyes into long polymer dye aggregates with non-conjugating bridges (superdyes) using monomer or aggregate sub-units can include switches, logic gates, wave splitters, interferometers, antennas (light harvesting or otherwise), and quantum logic gates. These constructs are preferably safe to use, cost effective build, and durable/easy to maintain.

Methods can be practiced which facilitate use, manufacture, assembly, maintenance, and repair of DNA templates of dyes into long polymer dye aggregates with non-conjugating bridges (superdyes) using monomer or aggregate sub-units which accomplish some or all of the previously stated objectives.

DNA templates of dyes into long polymer dye aggregates with non-conjugating bridges (superdyes) using monomer or aggregate sub-units can be incorporated into systems, such as quantum computers, which accomplish some or all of the previously stated objectives.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1C illustrate methods to create DNA-templated long polymer aggregates via chemical coupling of monomers or aggregate subunits. FIG. 1A shows dye coupling mediated by nucleic acid template. FIG. 1B shows dye coupling into a chain followed by tethering to DNA. FIG. 1C shows tethering the dyes as monomers to DNA template followed by the dye coupling.

FIG. 2A illustrates the general structural requirements for the dye monomer where the oval depicts a single dye or a superdye; FIG. 2B illustrates the general structural requirements for the bridge connecting the dyes; FIG. 2C illustrates the general structural requirements for specific functional groups to afford click-chemistry coupling; FIG. 2D illustrates the general structural requirements for examples of bacteriochlorin (BC1, BC2, and BC3), chlorin (CH1), squaraine (SQ), and bacteriochlorin superdye (bis-BC-1) equipped with azide/alkyne functional groups.

FIG. 3 illustrates established synthetic routes to install azide (from the first position to the sixth position) and alkyne (from the first position to the second position) functional groups for click-chemistry coupling. Deprotection of TIPS-ethynyl bacteriochlorin is shown (from the second position to the third position).

FIG. 4 illustrates routes toward monomers and aggregate subunits equipped with azide/alkyne functional groups (BC1 for Method 1 and 2; BC1-L for Method 3).

FIG. 5 illustrates the general strategy of DNA-mediated dye polymer synthesis. In the first to third steps, a solution of DNA with the first dye attached covalently is treated with a solution of free dyes. In each dye, one coupling group partner is protected to avoid coupling between the free dyes. In the fourth step, after the first reaction is complete, the unreacted free dye is removed from the reaction solution. In the fifth step, the solution is treated with deprotecting reagent followed by buffer exchange. The second to fifth steps are repeated until the sixth step, at which point the desired polymer length is achieved.

FIG. 6A illustrates a bacteriochlorin dye monomer equipped with alkyne and azide functional groups; FIG. 6B shows a Rosetta docking of the bacteriochlorin modified for the click-chemistry; FIG. 6C shows the best scoring structure of the DNA-bacteriochlorin with propargyl azide group located in the minor groove and alkyne group pointed outward; and FIG. 6D shows a DFT-optimized structure of the bacteriochlorin dimer fragment exhibiting excitonic coupling strength of 22.7 meV.

FIG. 7 and FIG. 7 (Continued) illustrate a proposed DNA-mediated coupling of squaraine dyes via an amide bond.

FIG. 8A shows synthesis and spectral properties of bis-BC; and FIG. 8B shows absorption and fluorescence of bis-BC and triazole-BC (as a monomer) in toluene at room temperature. Absorption in Q_{y} region was normalized at Q_{y} peak maximum. Fluorescence was corrected by absorptance. Transition dipole orientation is depicted with double-headed arrows.

FIG. 9 shows an established synthetic route to the covalently attached to DNA bacteriochlorin with a protected alkyne group.

FIG. 10 shows synthesis of bacteriochlorin linear array via click-chemistry as an example of array of aggregates synthesis.

FIG. 11 show enzyme-induced dye polymerization toward long J-aggregates.

FIG. 12 is a schematic representation of excitonic quantum-controlled basis change gate. The dots represent chromophores. The controlled basis change gate includes a phase shifting element between two basis change gates.

FIG. 13 shows the ¹H NMR spectrum of BC-L-NHS (CDCl₃, 600 MHz).

FIG. 14 shows the ¹³C NMR spectrum of BC-L-NHS (CDCl₃, 150 MHz).

FIG. 15 shows the ¹H NMR spectrum of bis-Br-BC (CDCl₃, 600 MHz).

FIG. 16 shows the ¹H NMR spectrum of BC-1 (CDCl₃, 600 MHz).

FIG. 17 shows the ¹³C NMR spectrum of BC-1 (CDCl₃, 150 MHz).

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

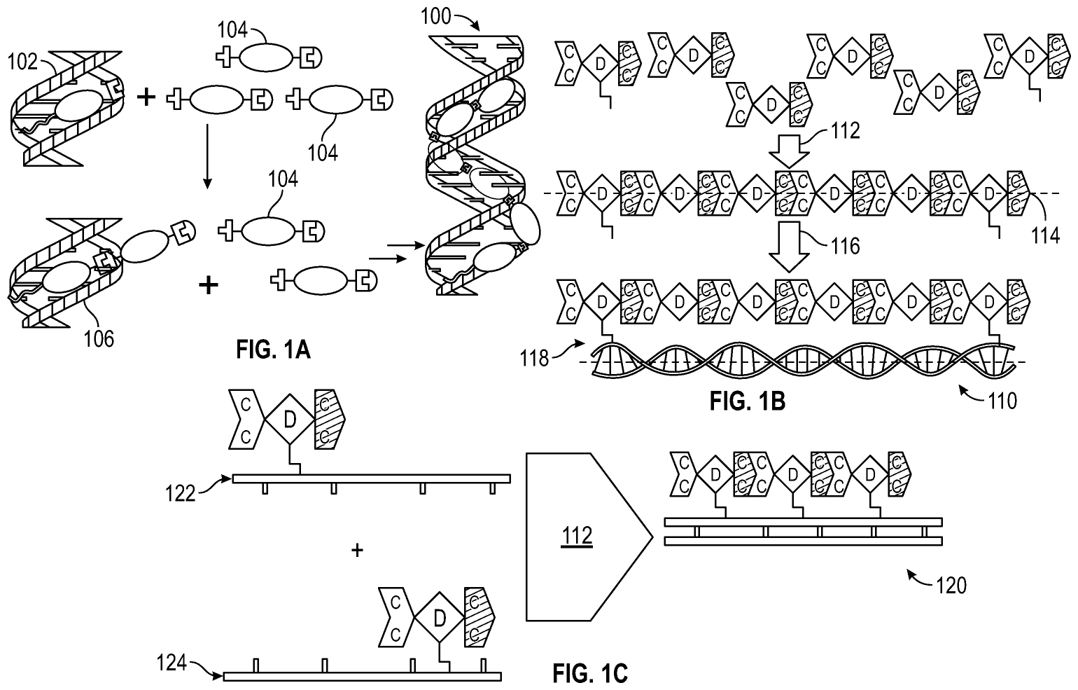

Coupling of dye monomers or aggregate sub-units via a chemical reaction is one of the approaches to create long polymer dye aggregates (other approaches include enzymatic and photo reactions). Using a chemical reaction, dyes D can be linked via couplers C into a polymer chain using the following general methods: (FIG. 1A: resulting in long polymer 100) dye 104 coupling mediated by the nucleic template (see dye attached to DNA 102) (dimer 106); (FIG. 1B: resulting in long polymer 110) dye coupling into a chain 114 as a result of addition 112 of a catalyst (e.g., light exposure or the addition of an enzyme), followed by the chain 114 tethering (via tethers 118) to the DNA template as a result of addition 116 of DNA, and (FIG. 1C: resulting in long polymer 120) tethering the dyes as monomers to DNA template followed by the dye coupling. In methods (2) and (3), the dye chain or dye monomers can be tethered to the same single strand, to opposing strands (e.g. first and second strands 122, 124) within one DNA duplex, or different DNA duplexes.

Click-chemistry chemical coupling is described herein. In this approach, the dyes are coupled via the copper(I)-catalyzed azide-alkyne cycloaddition (CuAAC) reaction between an azide and alkyne to form a 1,2,3-triazole ring linking the dyes. The dyes including superdyes can be coupled (1) in the presence of DNA; (2) prior to DNA templating, or (3) after the monomers are tethered to DNA. For (1) and (3), the copper catalyst might contain copper(I)-stabilizing ligands (e.g., tris(benzyltriazolylmethyl)amine, TBTA3) to prevent DNA damage (strand breaks) during CuAAC. A dye (or superdye) is modified with the azide-containing group and with a terminal alkyne group where the latter is protected with trialkyl silyl moiety [e.g., triisopropylsilylalkyne (TIPS-alkyne), trimethylsilyl alkyne (TMS-alkyne), t-butyldimethylsilylalkyne (TBDMS), thexyldimethylsilylalkyne (TDS-alkyne), benzyl dimethylsilyl alkyne (BDMS-alkyne), biphenyl dimethylsilyl alkyne (BDMS-alkyne), biphenyldiisopropylsilylalkyne (BDIPS-alkyne) and tris(biphenyl-4-yl)silyl (TBPS-alkyne)] in the form of a trialkyl silyl acetylene.

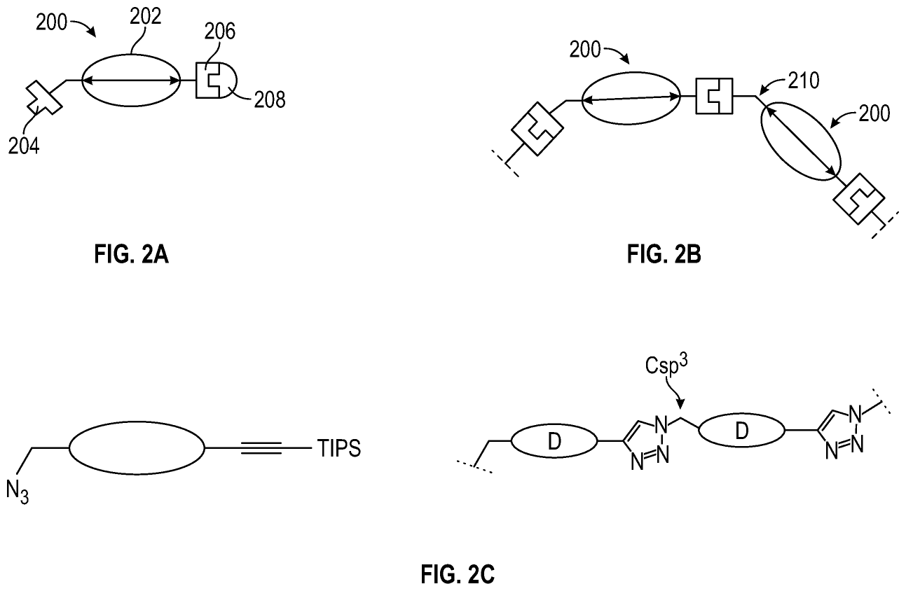

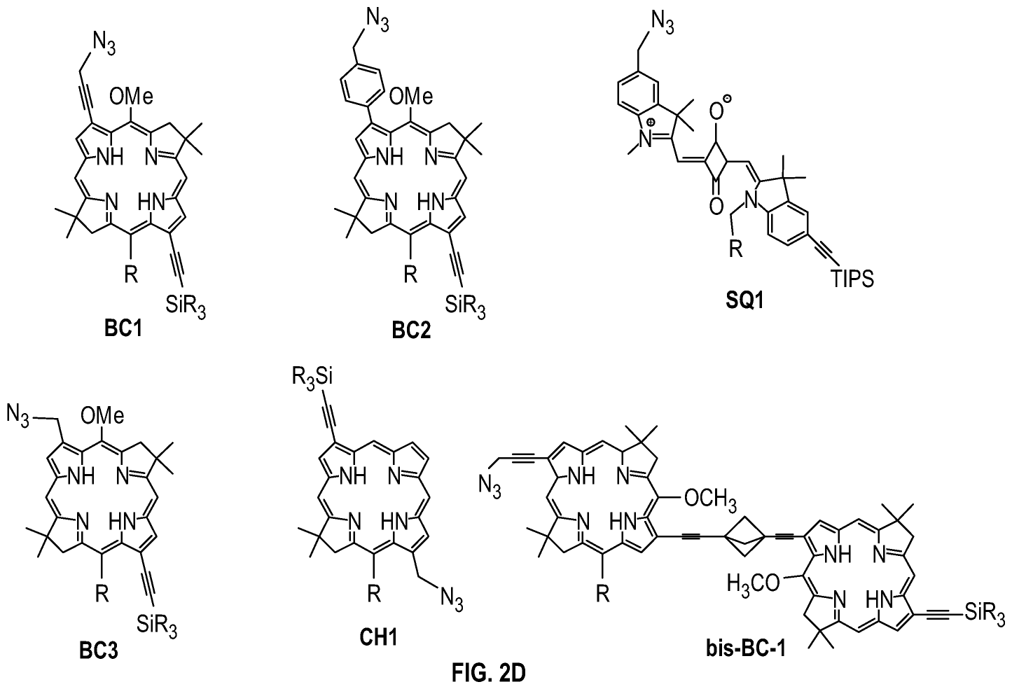

Any dye family can be used for dye coupling into a polymer via click-chemistry as long as the synthetic access to install coupling functional groups is available (FIGS. 2A-2D).

FIG. 2A illustrates the general structural requirements for the superdye 200. The superdye 200 includes a dye monomer 202, a first covalent coupler (CC) 204, a second covalent coupler (CC) 206, and a protecting group 208.

FIG. 2B illustrates a dimer fragment of the polymer chain, which clearly includes a non-conjugating bridge 210 for connecting the superdyes 200.

FIG. 2C illustrates the general structural requirements for specific functional groups to afford click-chemistry coupling. The triazole bridge is shown on the right side of FIG. 2C.

FIG. 2D illustrates the general structural requirements for examples of bacteriochlorin (BC1, BC2, and BC3), chlorin (CH1), squaraine (SQ), and bacteriochlorin superdye (bis-BC-1) equipped with azide/alkyne functional groups.

At least one coupling group should be protected to inhibit its chemical activity and prevent undesired self-coupling. For the methods shown in FIGS. 2A and 2C, the functional groups are thought to couple under DNA-compatible conditions using the protection-deprotection coupling strategy. As such, click-chemistry coupling is well suited for the dye monomer coupling into a polymer. The bridge 210 forming upon the coupling and connecting neighboring dyes in the polymer chain is non-conjugating and relatively short (3-6 Å) to ensure excitonic coupling between the dyes.

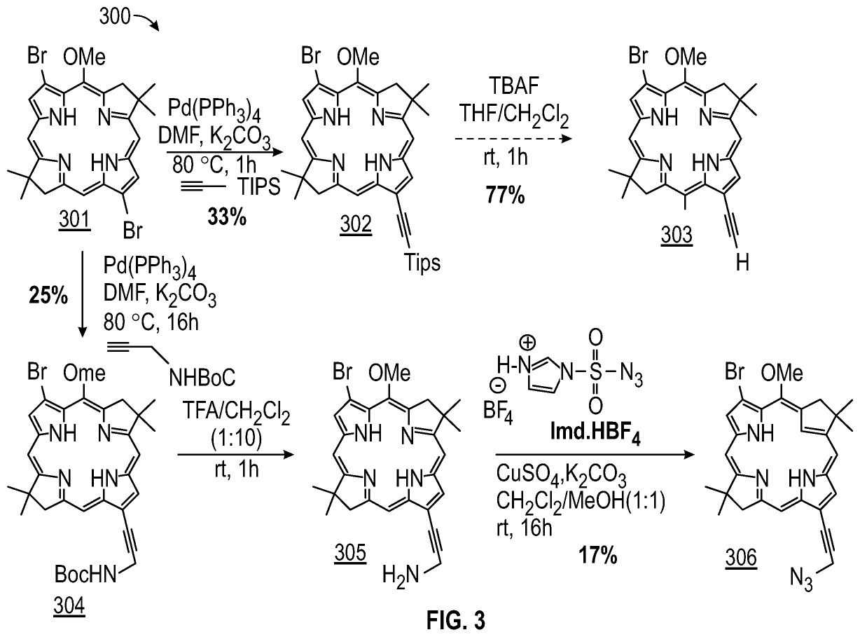

FIG. 3 illustrates established synthetic routes to install azide and alkyne functional groups for click-chemistry coupling. Deprotection of TIPS-ethynyl bacteriochlorin is also shown. The synthetic route to install azide functional group is shown from the first position 301 to the sixth position 306. The synthetic strategy to install ethyl functional group is shown from the first position 301 to the second position 302. The conditions to remove the protecting group are shown from the second position 302 to the third position 303.

The synthetic access to monomer dye equipped with azide and alkyne functional groups is described herein. To provide synthetic access to dye monomers and superdyes suitable for coupling via click-chemistry, we have established the synthetic routes 300 to independently install alkyne (FIG. 3, from positions 301 to 302) and azide groups (FIG. 3, from positions 301 to 306) starting with the 3,13-dibromobacteriochlorin building block at position 301. The terminal alkyne was protected with a triisopropylacetelyne group. The deprotection, critical for the dye polymer synthesis (see the click-chemistry procedure, infra), was tested at room temperature in the presence of TBAF and achieved in high yield (FIG. 3, from positions 302 to 303).

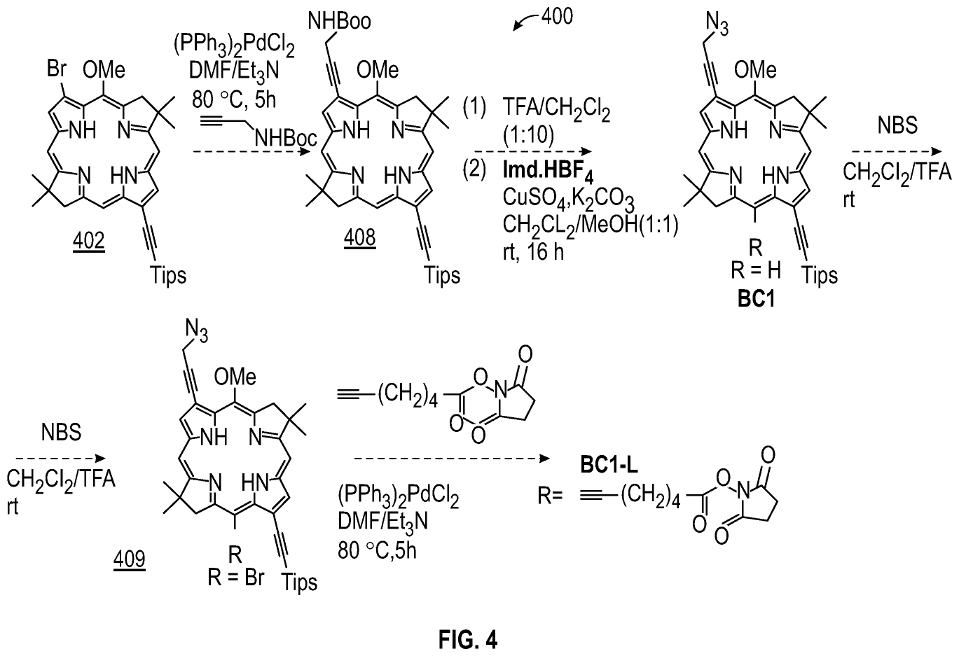

FIG. 4 illustrates future routes toward monomers and aggregate subunits equipped with azide/alkyne functional groups (BC1 for Method 1 and 2; BC1-L for Method 3). Synthesized BC1 is a specific example of a general structure 102 and a structure in FIG. 2C (left).

The next step is applying the established conditions to install alkyne and azide groups in a dye simultaneously (which we do not anticipate challenging as the disubstitution of 3,13-dibromobacteriochlorin with distinct functional groups is well established), and attach a covalent linker (FIG. 4). The linker for the DNA attachment in bacteriochlorin (position 409) can be added under the previously established conditions for an analogous bacteriochlorin modification.

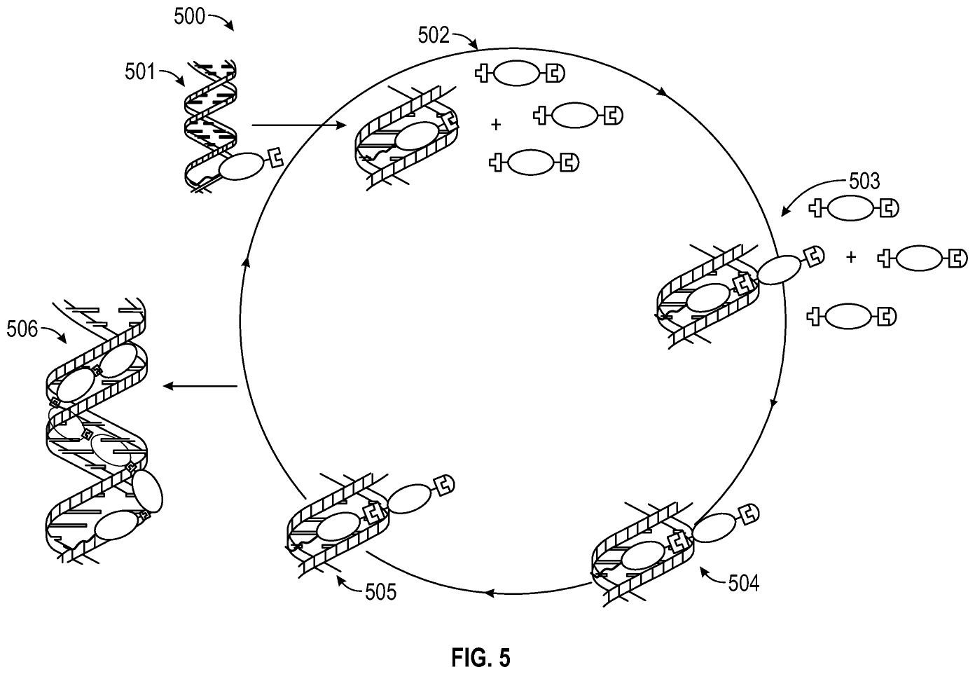

DNA-templated synthesis of dye polymer in solution via click-chemistry is described herein. A non-conjugated dye polymer is thought to be synthesized via successive coupling reactions of dye monomers on DNA. Repeated cycles of coupling and deprotection in an aqueous solution will enable precise control over the polymer length and dye sequence (FIG. 5: DNA-mediated dye polymer synthesis 500).

FIG. 5 illustrates the general strategy of DNA-mediated dye polymer synthesis. In the first to third steps 501-503, a solution of DNA with the first dye attached covalently is treated with a solution of free dyes. Specifically, in the first step 501, the first dye is covalently attached to dNA (a variety of linkers is possible). In the second step 502, free dyes are added to the solution and the protecting group to avoid self-reaction. In the third step 503, the click-chemistry catalyst is added. In each dye, one coupling group partner is protected to avoid coupling between the free dyes. In the fourth step 504, after the first reaction is complete, the unreacted free dye is removed from the reaction solution. In other words, the unreacted free dye is removed. In the fifth step 505, the solution is treated with deprotecting reagent followed by buffer exchange. In other words, the functional group is deprotected. The second to fifth steps 502-503 are repeated until the sixth (end) step 506, at which point the desired polymer length is achieved.

To provide the rigidity to the dye polymer vital for long-range exciton delocalization, the growing polymer chain will be routed along a DNA minor groove. For a head-to-tail dye orientation, each dye should be equipped with two coupling groups on opposite sides of the dye's transition dipole moment. Thereof, the method will afford a long aggregate of dyes (i.e. a dye polymer) where dyes are covalently linked to each other and align in a head-to-tail fashion. DNA is a solid support for polymer growth and a template to rigidify the polymer chain, minimizing its motion.

The first dye will be covalently attached to the oligo strand termini (3′ or 5′ end) or internally via various linkers. The attachment of the first dye can be carried out during oligonucleotide solid phase synthesis (e.g., via phosphoramidite method) or post-modification. The dye polymer growth can be carried out on dsDNA (FIG. 5). Alternatively, the dye polymer growth is carried out with a single-stranded DNA, RNA, or oligonucleotide in solution or attached to a solid phase. When the desired length of a dye polymer is achieved, the nucleic acid single strand is hybridized with a complementary strand.

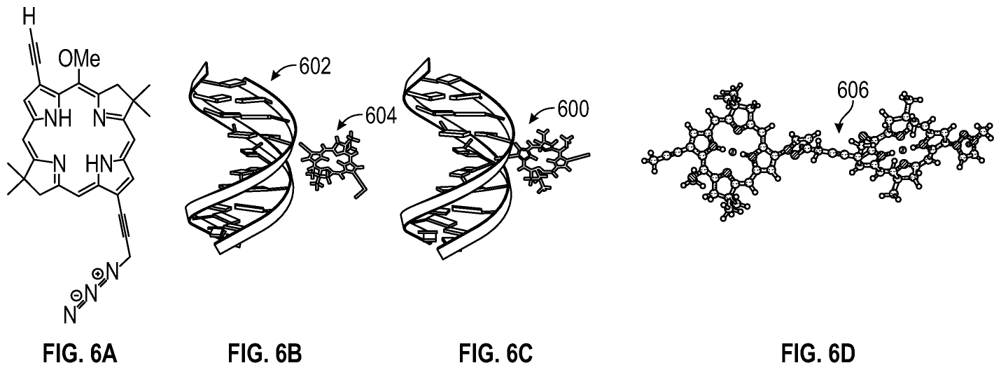

FIG. 6A illustrates a bacteriochlorin dye monomer equipped with alkyne and azide functional groups. FIG. 6B shows a Rosetta docking of the bacteriochlorin 602 modified for the click-chemistry 604. FIG. 6C shows the best scoring structure 600 of the DNA-bacteriochlorin 602 with propargyl azide group located in the minor groove and alkyne group pointed outward. FIG. 6D shows a DFT-optimized structure of the bacteriochlorin dimer fragment 700 exhibiting excitonic coupling strength of 22.7 meV.

The photophysical properties of the monomer and dimer unit for Method 1 are described herein. A candidate bacteriochlorin dye (FIG. 6A) was examined by computational methods to evaluate its suitability for the proposed method of DNA-mediated dye polymer growth in the minor groove (Method 1). Docking of the monomer using Rosetta software demonstrated that the bacteriochlorin associates with the minor groove via propargyl azide. At the same time, the alkyne group is pointed outward, being available for the coupling with a monomer in solution (FIGS. 6B-6D). Density-functional-theory calculations of the polymer dimer fragment predicted the coupled dyes to maintain head-to-tail orientation and exhibit excitonic coupling strength of 22.7 meV.

Click-chemistry procedure(s) is/are described herein.

A proposed procedure for click reaction in aqueous solution (for Method 1) is described herein. A DNA labeled with the first monomer dye is placed in a 1.5 mL vial. In a separate vial, CuBr solution (100 mM in DMSO/tBuOH 3:1) and dye monomer solution (100 mM in DMSO/tBuOH 3:1) are vortexed and added to the DNA. The resulting solution is shaken at 25° C. for several hours and evaporated to dryness. Sodium acetate solution (0.3 M) is added, and the suspension is incubated for 1 h. Next, 1 mL ethanol is added, the vial vortexed, and placed in a freezer (−20° C.) overnight. After centrifugation, the supernatant is carefully removed from the DNA pellet. Next, 70% ethanol (−20° C.) is added, the vial is vortexed, centrifuged, and the supernatant is removed. This washing step is repeated twice, after which the pellet is subjected to a deprotection step.

Regarding the deprotection of the TIPS-alkyne, a pelleted DNA is dissolved in dry acetonitrile/DMF (4:1 v/v. TBAF (1.0 M in THF) is added dropwise, and the solution is shaken at 45° C. for several hours. Excess fluoride ions are quenched with MeOTMS. For the next click reaction, the organic solvent is exchanged for water: the reaction solution is evaporated to near dryness. Water is added, the solution is frozen, lyophilized to dryness, and redissolved in an appropriate amount of water to repeat the click reaction in the solution step.

A proposed procedure for click reaction in organic solvent (for Method 2) is described herein. The first dye is immobilized on a solid support such as resin. The growth of the polymer chain is accomplished via a successive cycle of CuAAC and TIPS deprotection in organic solvent.

A proposed procedure for click reaction in aqueous solution (for Method 3) is described herein. A DNA labeled with the azide/alkyne(deprotected) monomer(s) is placed in a 1.5 mL vial. A CuBr solution (100 mM in DMSO/tBuOH 3:1) is added to the DNA. The resulting solution is shaken at 25° C. for several hours and evaporated to dryness. Sodium acetate solution (0.3 M) is added, and the suspension is incubated for 1 h. Next, 1 mL ethanol is added, the vial vortexed, and placed in a freezer (−20° C.) overnight. After centrifugation, the supernatant is carefully removed from the DNA pellet. Next, 70% ethanol (−20° C.) is added, the vial is vortexed, centrifuged, and the supernatant is removed. This washing step is repeated twice, after which the pellet is redissolved in the buffer of interest.

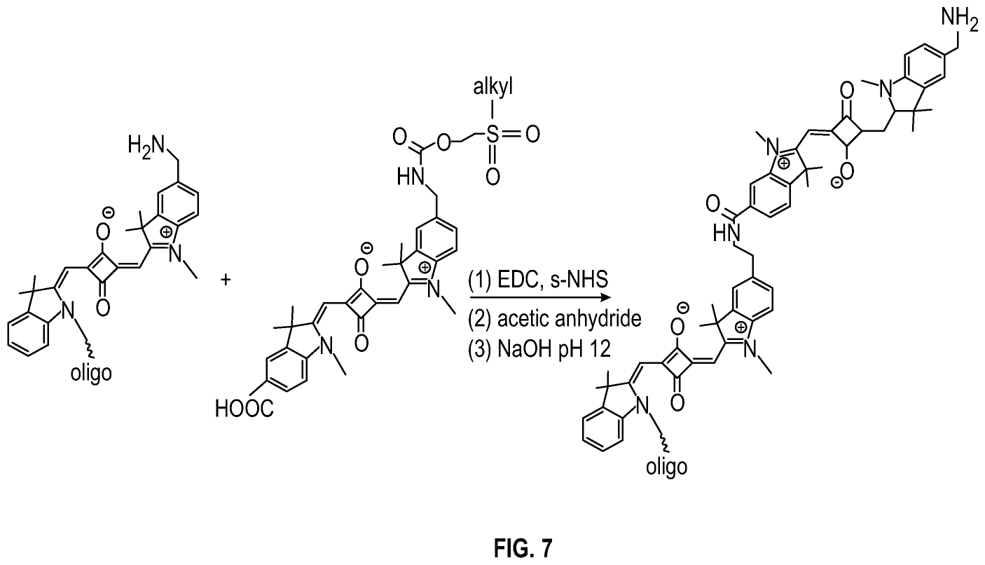

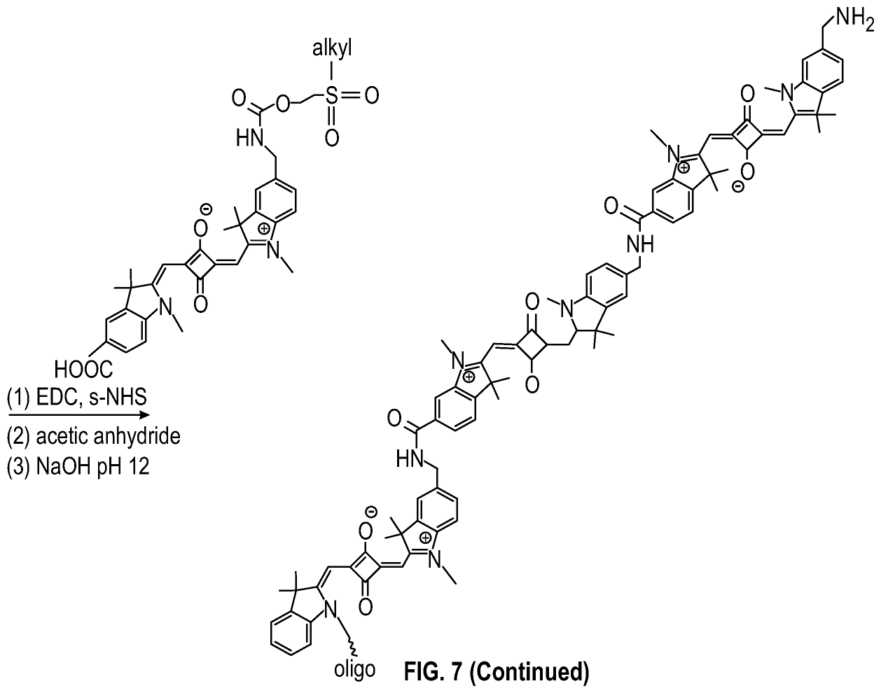

The dye coupling uses an amide bond. In this method, the dye is equipped with a carboxylic ester group (or activated carboxylic ester) on one end and a primary amine on the opposite end (FIG. 7 and FIG. 7 (Continued)).

Frenkel Exciton Theory, otherwise known as Molecular Exciton Theory, describes exciton delocalization and exciton-exciton interaction in molecular aggregates. We describe the key parameters present in the Frenkel Exciton model that include J (excitonic hopping parameter), which is based on the transition dipole moment (m) of each dye and describes the delocalization of a single exciton in a wave-like manner over a DNA-templated aggregate; the less known, yet fundamentally crucial, K (biexciton interaction energy), which is induced solely by the difference in static dipole moment (Δd) of each dye; and A, the single site exciton-exciton interaction energy (interaction energy of two excitons both on one site). The reader should note that the ‘J’ here is distinct from that of the J designating the FRET overlap integral above, and is typically utilized in subsequent context as ‘J_{m,n}’ where the subscripts denote dye positions (see below). Because excitons have bosonic properties, they can be described as either hard core (i.e., two excitons cannot reside on the same dye) or soft-core bosons (i.e., two excitons can reside on the same dye). The aim of this section is to describe the details of J, K, and A and highlight their potential utility within the context of this discussion.

Molecular dye aggregates can form Frenkel excitons, otherwise known as molecular excitons, which are delocalized excitons and are also known as delocalized collective excitations. The Frenkel excitons are shared between each of the dyes in the aggregate in a wave-like manner. These delocalized excitons or molecular or Frenkel excitons exhibit unique optical properties. Note that exciton states that exhibit exciton delocalization are created by the interaction of two (or more) strongly coupled dyes resulting in excited states that are split (i.e., Davydov splitting) as compared to the monomer state.

An augmented Frenkel Hamiltonian approximates the behavior of Frenkel excitons and is given by:

$${\hat{H}}^{( e )} = {\sum }_{m} {ε}_{m}^{e} {\hat{B}}_{m}^{†} {\hat{B}}_{m} + {\sum }_{m , n}^{m \neq n} {J}_{m , n} {\hat{B}}_{m}^{†} {\hat{B}}_{n} + \frac{1}{2} {\sum }_{m , n}^{m \neq n} {K}_{m , n} {\hat{B}}_{m}^{†} {\hat{B}}_{n}^{†} {\hat{B}}_{m} {\hat{B}}_{n} + \frac{1}{2} {\sum }_{m , n}^{m \neq n} {Δ}_{m} {\hat{B}}_{m}^{†} {\hat{B}}_{m}^{†} {\hat{B}}_{m} {\hat{B}}_{m}$$

Note that εₘ^{e} is the monomer transition energy of a single excited dye (excited monomer: S₀→S₁) on the m site, \hat{B}ₘ^{†}(\hat{B}ₘ) is the bosonic exciton creation (annihilation) operator on site m. K_{m,n} is the exciton-exciton interaction energies and is related to the average energy of two singly excited dyes, one each on dye sites m and n.

The excitonic hopping parameter, J_{m,n}, is the strength of that component of the Coulombic coupling (electrodynamic interactions) between dye m and dye n (within a molecular aggregate) that induces a transition from the excited state of one molecule while simultaneously inducing a transition from the ground state to the electronic excited state of the other molecule. Thereby, an excitation is transferred from one molecule to the other. This is a hopping interaction and can be referred to in a number of categorical ways in terms of interaction, hopping, and coupling or in the context of a parameter, shift, or potential. Relative to the former, J_{m,n} is referred to as an exchange interaction, instantaneous intermolecular interaction, resonant interaction or coupling, resonant exciton hopping, intermolecular Coulombic interaction, intermolecular dipole-dipole coupling, long-range dipole-dipole Coulombic interaction, excitonic coupling, and electronic interaction. In the context of the latter, J_{m,n} is called a hopping parameter, monoexcitonic shift, and point dipole interaction potential. Although not a complete list, the number of ways J_{m,n} is described suggests that a more thorough description may be useful. The excitonic hopping interaction inherent in J_{m,n} enables exciton delocalization and relaxation. Where intermolecular distances are less than ˜4 Å (e.g., aggregates with π-stacking or covalently bridged), wave function overlap can induce intermolecular charge transfer (also described as superexchange coupling or Dexter mechanism, coupling or interaction) and can contribute to the J_{m,n} term.

J_{m,n} is often approximated as the coupling between a pair of transition point dipoles. The point dipole approximation is inadequate because the spacing between the dyes is shorter than their length. Instead, an extended dipole approximation was employed where the dipoles are modeled as two-point charges of opposite sign separated by nearly the length of the dye core. This extended dipole approximation better accounts for the physical charge distribution that spreads out over the length of the molecule with the maxima of charge density occurring near the ends of the dye.

J_{m,n} enables exciton delocalization, the delocalization of a single exciton between dyes m and n. The delocalization is a result of transition dipole of dye m coupling with the transition dipole on dye n (Coulombic coupling or electrodynamic interactions) upon excitation of the dyes by an electromagnetic field (e.g., light). The strength of that coupling is J_{m,n}. Another way to describe exciton delocalization is that a resonant exciton hopping results due to the dipole-dipole coupling between dyes m and n. This coupling induces an electronic transition in one dye from the excited state to the ground state while simultaneously inducing an electronic transition in the other dye from the ground state to the excited state. Note that the Coulombic coupling, or electrodynamic interactions, between the dyes in the aggregate leads to the sharing of excited state energies between all dyes in the aggregate and the sharing of the excited state energies allows sharing, or the delocalization, of the exciton between dyes in the aggregate (i.e., dyes m and n) and thus there is a J_{m,n} for each dye pair, m and n, in the aggregate. J_{m,n} can be mathematically described, with certain approximations (see references below for descriptions of the approximations), in the following manner:

$${J}_{m , n} = \frac{1}{4 π ε {ε}_{o}} ( \frac{{μ}_{m} \cdot {μ}_{n}}{{\vert {R}_{m , n} \vert}^{3}} - 3 \frac{( {μ}_{m} \cdot {R}_{m , n} ) ( {μ}_{n} \cdot {R}_{m , n} )}{{\vert {R}_{m , n} \vert}^{5}} )$$

where μₘ and μₙ are the transition dipoles of dyes m and n and R_{m,n} is the vector connecting dyes m and n. J_{m,n} is inversely proportional to the cube of R_{m,n} with typical R_{m,n} values between dyes in the aggregate ranging from 2 nm or less in order to induce exciton delocalization. If exciton delocalization is not present, the dyes act independently (do not share excited states) and an aggregate does not exist. Thus, the behavior of the aggregate in the excited state differs and is unique from the behavior of single, or monomeric, dyes.

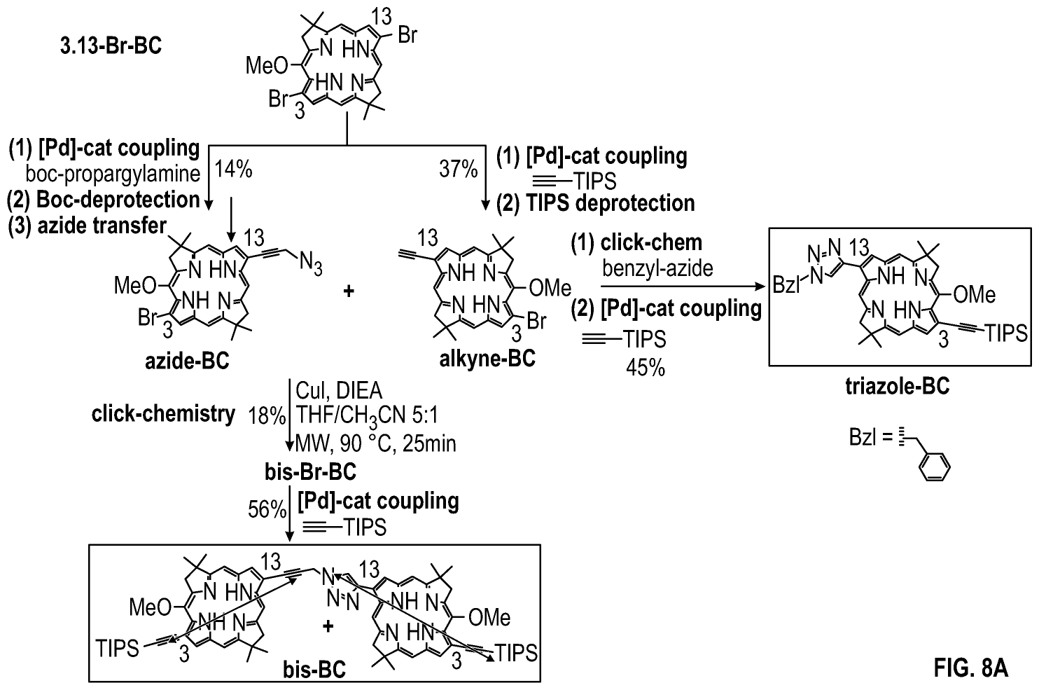

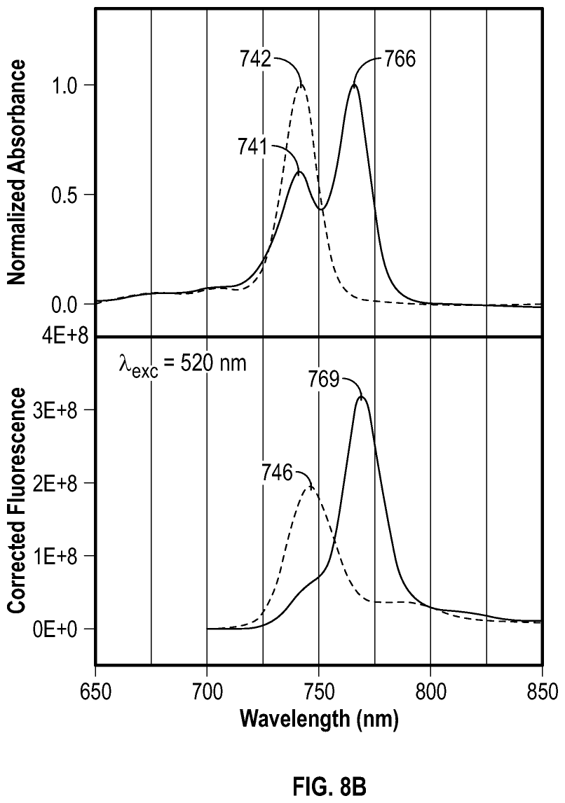

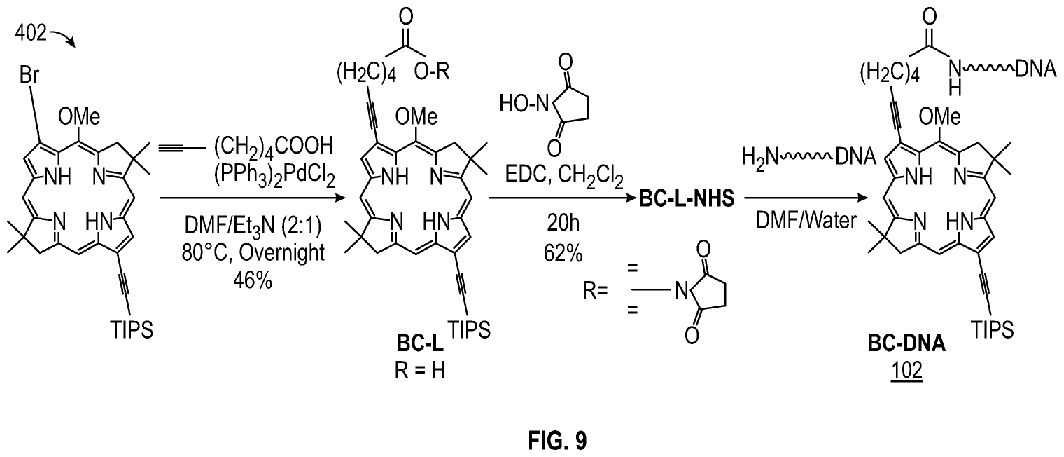

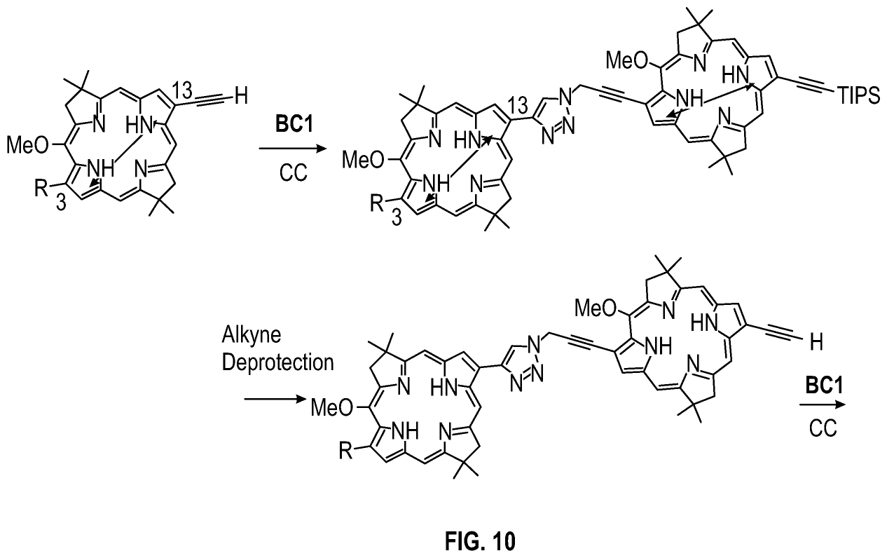

It is desirable for the dye aggregates for excitonic systems to be emissive. J-aggregates (i.e., dyes are stacked head-to-tail) are the most emissive of aggregates. In general, it is challenging to create a J-aggregate, as organic dyes, when forming an aggregate, tend to orient their surfaces co-facially to benefit from stabilizing 71-71 effect. The tendency to form H-aggregate is especially pronounced in the aqueous environment where organic dyes driven by hydrophobic effect prompt to minimize their surface exposure to water. To overcome this challenge, we recently created a Bchl dimer bis-BC with two dyes bridged head-to-tail via a covalent bond. In contrast to previously reported dyads with a conjugating bridge, in dimer bis-BC, the methylene bridge prevents the conjugation of two chromophore systems. To synthesize bis-BC, we developed synthetic routes to alkyne-BC and azide-BC (FIG. 8A). Next, the dyes were coupled by means of copper-catalyzed click-chemistry reaction under the microwave with the yield of 18%. The resulting bis-BC exhibited spectral properties of the J-dimer. Namely, the bis-BC absorption and fluorescence were red-shifted relative to the monomer's absorption and fluorescence (FIG. 8B). In addition, the bis-BC was significantly fluorescent. From the observed spectral shift, the J_{1,2}, was estimated to be ˜30 meV, which is in accordance with J_{1,2} in the natural Bchla dimers in photosynthetic antenna. The bis-BC represents an elemental unit of dye polymer. We anticipate that extending (“growing”) the bis-BC into a long polymer will further increase J_{1,2} and fluorescence.

Synthesized bis-BC is a specific example of a general structure 106 (without attachment to DNA) and a specific example of structure FIG. 2C (right). The bis-BC contains two distinct transition dipole moments.

Acquired spectral properties (absorption and fluorescence) shown in FIG. 8B confirm the presence of exciton delocalization in bis-BC. Click-chemistry reaction used to prepare bis-BC is a specific example of covalent coupling chemical reaction to be used in an array of aggregates synthesis.

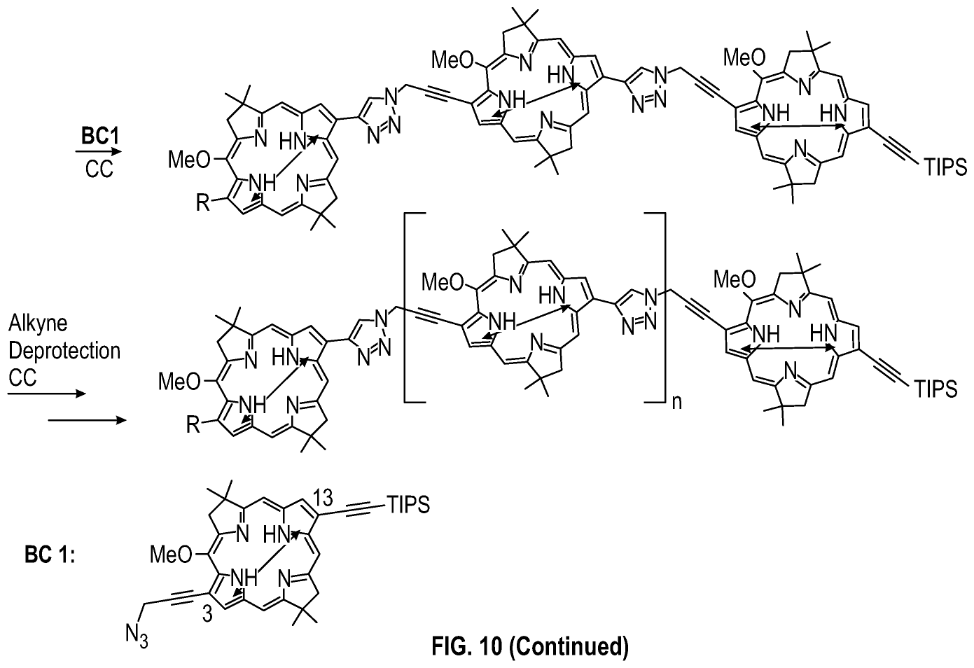

The R in FIG. 10 represents Hal (e.g., see position 303), linker (BC-L), oligio (e.g., BC-DNA), peptide, solid support or other substituent unreactant with azide or alkyne.

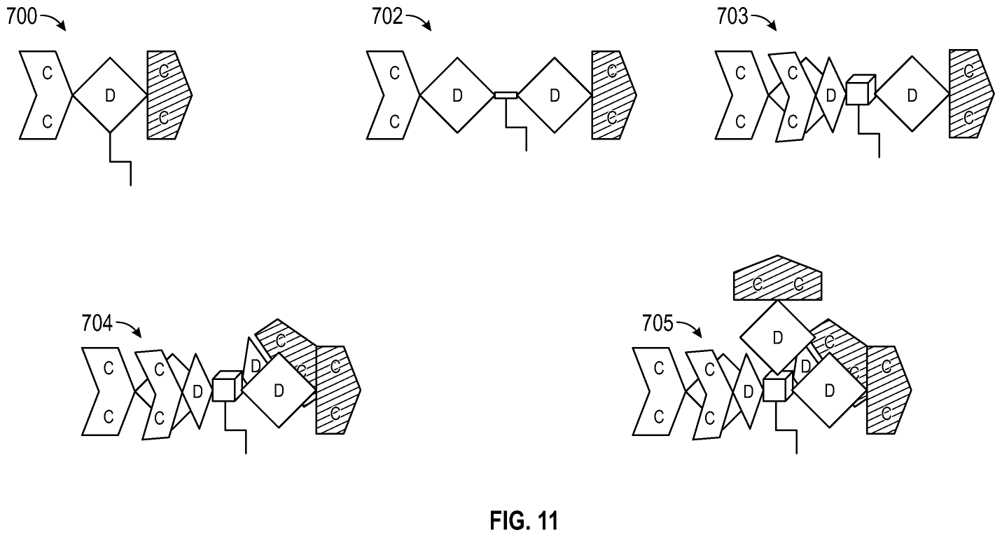

FIG. 11 shows superdyes that can be used as building blocks for more complex junctions. As shown, the junction superdyes 200 can form dyads 702, triads 703, tetrads 704, pentads 705, hexads, heptads, octads, etc. These complex junctions can be used to form one-dimensional transmission lines and excitonic wires, two dimensional excitonic circuits, and even three-dimensional excitonic circuits. The junctions may or may not be supported with external templates and physical support structure(s).

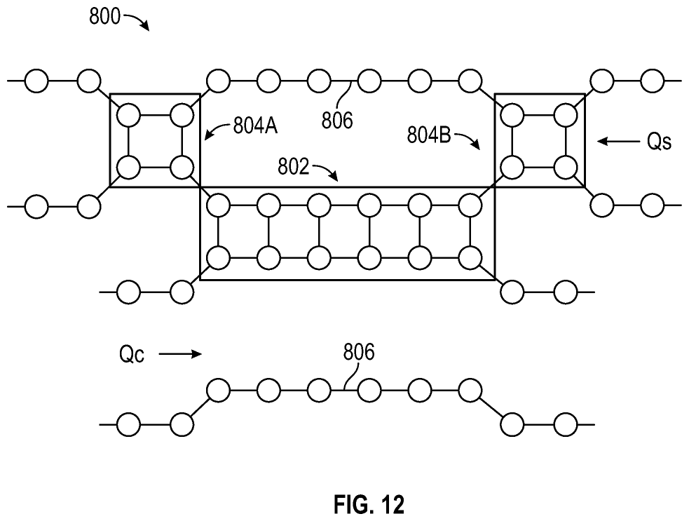

The controlled basis-change gate 1000 of FIG. 12 enables the entanglement of many-exciton states so that a network of quantum gates as if it is performing many different computations simultaneously. The exemplary controlled basis-change gate 1000 can be made from two basis-change gates 1004 and a phase-shift gate 1002, with the phase-shift gate between the two basis-change gates. The controlled basis-change gate 1000 relies on the interaction between two excitons. When two excitons reside on neighboring chromophores they feel each other's presence just like two electrons will feel each other's Coulomb repulsion when they are brought close together. The two-exciton interaction arises from static Coulomb interactions between molecules and is strong when the molecules have an asymmetric molecular structure than those with a symmetrical molecular structure. Asymmetric molecules possess a permanent electric dipole which changes sign when the molecule is excited from the ground state to the excited state. The static Coulomb interaction, in this case is a dipole-dipole interaction which, when both chromophores are excited (the two-exciton case), differs in sign from the case when only one chromophore is excited (the one-exciton case). Due to the static Coulomb interactions between chromophores one exciton will accumulate extra phase in the presence of the other exciton. As a result, the presence or absence of one exciton can control of the other exciton moves through a basis change gate.

Additionally, additional gates may be incorporated into the architecture, such as, but not limited to, Hadamard gates, momentum switches, and CO gates.

Using the DNA architecture to control the positioning of the chromophores and the wide range of chromophores with different optical transition energies, the phase-shift gates can be controlled to only have a finite set of phase angles. By controlling the position and optical transition energies of the various gates, a set of gates, or a quantum circuit.

The wires, gates, and switches as discussed above can be joined together to answer questions that benefit from quantum algorithms such as, but not limited to, sorting, factoring, and database searching. To initialize the system, input chromophores are excited by using the light with the proper wavelength and polarization in such a manner that only the desired subset of chromophores is excited when the system is hit with an initializing pulse of light. The wavelength and degree of polarization of the light may match that of input chromophores in order to excite the chromophores and cause exciton emission. Any light source that may produce the wavelength used by the chromophore may be used, such as, but not limited to, lasers, including ultra-fast lasers.

After initializing, the excitons then propagate from chromophore to chromophore along the wires into the various gates. The various gates than calculate the answer, such as a sorted list or mathematical problem. The output, or readout, can be done by using fluorescent reporter dyes to which the answer of the quantum computation is delivered by ordinary FRET. While this would be particularly beneficial for problems in which the output has a limited number of bits, these problems have applications in aeronautics, Earth and space sciences, and space exploration, among other fields of research. Additionally, these systems because it demonstrates that quantum coherence is observed at room temperature in a wet and noisy environment, an environment that is normally hostile to quantum coherence.

From the foregoing, it can be seen that the present disclosure accomplishes at least all of the stated objectives.

### Methods

General Experimental Section. ¹H NMR spectra (300, 600 MHz) were collected at room temperature in CDCl₃. Chemical shifts (6) were calibrated using CDCl₃ ¹H residual proton signals at 7.26 ppm. All solvents and commercially available reagents were used as received unless otherwise specified.

3-Bromo-5-methoxy-8,8,18,18-tetramethyl-13-(triisopropylsilyl)ethynyl-bacteriochlorin (see e.g., position 302). A sample of 3,13-dibromo-5-methoxy-8,8,18,18-tetramethylbacteriochlorin (see e.g., position 301) (30.0 mg, 0.053 mmol) and potassium carbonate (73.3 mg, 0.53 mmol) in N,N-dimethylformamide (5.3 mL) was degassed via three freeze-pump-thaw in an oven-dried 25 mL Schlenk flask equipped with a stir bar. The flask was backfilled with argon and allowed to warm to room temperature. (Triisopropylsilyl)acetylene (23 μL, 0.106 mmol), tetrakis(triphenylphosphine)palladium (12.3 mg, 0.0106 mmol), and copper (I) iodide (2.0 mg, 0.0106 mmol) were added, and the reaction mixture was stirred at 80° C. for 90 min in the sealed flask. An additional portion of (triisopropylsilyl)acetylene (23 μL, 0.106 mmol), tetrakis(triphenylphosphine)palladium (6.2 mg, 0.0053 mmol), and copper (I) iodide (1.0 mg, 0.0053 mmol) were added at room temperature, after which the mixture was stirred at 80° C. for 30 min in the sealed flask. The reaction mixture was diluted with ethyl acetate. The organic layer was washed with water (50 mL) and brine (50 mL), dried with sodium sulfate and concentrated using a rotary evaporator. The crude was purified by column chromatography [silica, dichloromethane/n-hexanes (1:2) to (1:1)] to afford the desired product as a green solid (11.5 mg, 33%). ¹H NMR (CDCl₃, 300 MHz) δ −1.89 (bs, 1H), −1.71 (bs, 1H), 1.36 (s, 3H), 1.37 (s, 18H), 1.93 (s, 12H), 4.34 (s, 3H), 4.40 (s, 4H), 8.48 (s, 1H), 8.53 (s, 1H), 8.68 (d, J=2.2 Hz, 1H), 8.76 (s, 1H), 8.88 (s, 1H); λ_{abs}.: 356, 375, 516, 738 nm.

3-Bromo-13-ethynyl-5-methoxy-8,8,18,18-tetramethyl-bacteriochlorin (see e.g., position 303). A sample of 3-bromo-5-methoxy-8,8,18,18-tetramethyl-13-(triisopropylsilyl)ethynyl-bacteriochlorin (see e.g., position 302) (11.5 mg, 0.017 mmol) was dissolved in anhydrous tetrahydrofuran (3.5 mL) in an oven-dried 10 mL round-bottom flask under argon, and tetrabutylammonium fluoride was added (1 M in THF, 0.034 mL, 0.034 mmol). The solution was stirred for one h at room temperature. The reaction mixture was diluted with dichloromethane (35 mL), washed with water (40 mL) and brine (35 mL), dried with sodium sulfate, and concentrated to afford a green solid (6.6 mg. 77%). ¹H NMR (CDCl₃, 600 MHz) δ −1.99 (bs, 1H), −1.76 (bs, 1H), 1.93 (s, 6H), 1.95 (s, 6H), 3.87 (s, 1H), 4.34 (s, 3H), 4.40 (s, 2H), 4.42 (s, 1H), 8.50 (s, 1H), 8.57 (s, 1H), 8.71 (d, J=2.5 Hz, 1H), 8.80 (d, J=2.0 Hz, 1H), 8.90 (s, 1H).

3-Bromo-5-methoxy-13-[3-(tert-butoxycarbonylamino)propynyl]-(8,8,18,18-tetramethyl-bacteriochlorin (see e.g., position 304). Following a reported procedure with modifications, 3,13-dibromo-5-methoxy-8,8,18,18-tetramethylbacteriochlorin (see e.g., position 301) (60.0 mg, 0.107 mmol), N-Boc-propargylamine (18.3 mg, 0.118 mmol), and potassium carbonate (148.4 mg, 1.07 mmol) were added under argon to an oven-dried 50-mL Schlenk flask equipped a stir bar. N,N-dimethylformamide (10.8 mL) was added, and the mixture was degassed via three freeze-pump-thaw cycles. The flask was backfilled with argon and allowed to warm to room temperature. Tetrakis(triphenylphosphine)palladium (24.7 mg, 0.021 mmol) was added, and the sealed flask was heated to 80° C. After 16 h, the solution was diluted with ethyl acetate (70 mL). The organic layer was washed with water, and brine, dried with anhydrous sodium sulfate, and concentrated using a rotary evaporator. The crude solid was purified by column chromatography (silica, EtOAc/toluene (1:20) to (1:2)), followed by column chromatography (silica, EtOAc/n-hexanes (1:5) to (1:2)) to afford the desired product as a green solid (18.9 mg, 25%). ¹H NMR (CDCl₃, 300 MHz) δ −1.96 (bs, 1H), −1.76 (bs, 1H), 1.58 (s, 9H), 1.92 (s, 6H), 1.94 (s, 6H), 4.34 (s, 3H), 4.40 (s, 2H), 4.42 (s, 2H), 4.58 (bd, J=3.1 Hz, 2H), 8.50 (s, 1H), 8.55 (s, 1H), 8.708 (d, J=1.9 Hz, 1H), 8.715 (s, 1H), 8.85 (s, 1H); λ_{abs}: 358, 371, 509, 733.

3-Bromo-5-methoxy-13-(3-amino)propynyl-8,8,18,18-tetramethyl-bacteriochlorin (see e.g., position 305). A sample of 3-bromo-5-methoxy-13-[3-(tert-butoxycarbonylamino)propynyl]-(8,8,18,18-tetramethyl-bacteriochlorin (see e.g., position 304) (6.2 mg, 0.01 mmol) was dissolved in dichloromethane (1.3 mL) in a 7-mL conical vial under argon and was stirred for 2 min. Trifluoroacetic acid (0.262 mL, 3.43 mmol) was added dropwise, and the solution was stirred for 1 h under argon. The solution was dissolved in ethyl acetate (20 mL). The organic layer was washed with saturated aqueous sodium bicarbonate (25 mL), water (30 mL), and brine (25 mL), dried with sodium sulfate and concentrated. The crude green solid (5.9 mg) was used without further purification. ¹H NMR (CDCl₃, 600 MHz) δ −1.92 (bs, 1H), −1.74 (bs, 1H), 1.91 (s, 6H), 1.92 (s, 6H), 4.09 (s, 2H), 4.33 (s, 3H), 4.38 (s, 2H), 4.40 (s, 2H), 8.48 (s, 1H), 8.52 (s, 1H), 8.681 (d, J=2.4 Hz, 1H), 8.685 (s, 1H), 8.84 (s, 1H).

3-Bromo-5-methoxy-13-(3-azido)propynyl-(8,8,18,18-tetramethyl-bacteriochlorin (see e.g., position 306). A solution of 3-bromo-5-methoxy-13-(3-amino)propynyl-8,8,18,18-tetramethyl-bacteriochlorin (see e.g., position 305) (5.0 mg, 0.009 mmol), potassium carbonate (34.0 mg, 0.246 mmol), copper (II) sulfate pentahydrate (1.75 mg, 0.007 mmol), and imidazole-1-sulfonyl azide tetrafluoroborate (2.35 mg, 0.009 mmol) in dichloromethane/methanol (1:1, v/v; 0.62 mL) was stirred in a 1-mL conical vial under argon at room temperature for 16 h. The mixture was poured into ethyl acetate (20 mL), washed with water (25 mL), dried with anhydrous sodium sulfate, and concentrated. The crude was purified by column chromatography [silica, ethyl acetate/n-hexanes (1:3) to (1:2)] to afford a green solid (0.9 mg, 17%). ¹H NMR (CDCl₃, 600 MHz) δ −1.99 (bs, 1H), −1.75 (bs, 1H), 1.93 (s, 6H), 1.94 (s, 6H), 4.34 (s, 3H), 4.40 (s, 2H), 4.42 (s, 2H), 4.56 (s, 2H), 8.50 (s, 1H), 8.58 (s, 1H), 8.72 (d, J=2.4 Hz, 1H), 8.78 (d, J=1.9 Hz, 1H), 8.89 (s, 1H); λ_{abs} 361, 512, 734 nm.

Computational Methods. To test the docking protocol, a bacteriochlorin dye depicted in FIG. 6A was docked into a 10 bp DNA duplex with a semi-randomly chosen sequence. The bacteriochlorin dye was arbitrarily placed near the minor groove of the DNA as the initial configuration (FIG. 6B). Next, a reported docking protocol with modifications was used to obtain 200 docked dye-DNA configurations, the best scoring of which is shown in FIG. 6C. The protocol involved randomly translating and rotating the dye to obtain a starting orientation. In the next step, the dye was translated towards the DNA, after which a high-resolution docking step was performed to finalize the dye orientation. Finally, energy minimization was performed to obtain the final scored configuration.

bis-BR-BC

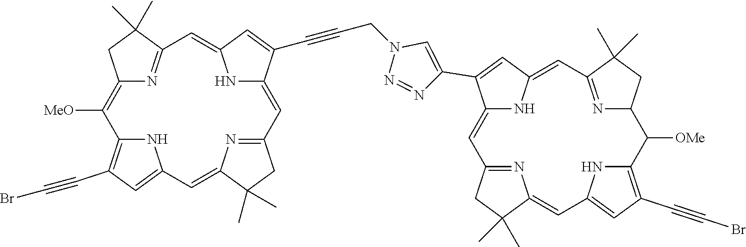 [SMILES: COc1c2nc(cc3cc(C#CCn4cc(-c5c/c6[nH]/c5=C\C5=NC(=C\c7cc(C#CBr)c([nH]7)C(OC)C7CC(C)(C)C(=N7)\C=6)/C(C)(C)C5)nn4)c(cc4nc(cc5cc(C#CBr)c1[nH]5)C(C)(C)C4)[nH]3)C(C)(C)C2]

3-Bromo-5-methoxy-13-(3-(4′-(11-Bromo-9-methoxy-6,6,16,16-tetramethyl-bacteriochlorin)-(1-H-1,2,3-triazol-1′-yl) propynyl)-(8,8,18,18-tetramethyl-bacteriochlorin (bis-Br-BC). Samples of azide-BC (4.0 mg, 0.0071 mmol), alkyne-BC (4.0 mg, 0.0079 mg), diisopropylethylamine (0.0036 mmol, 0.356 mL of 10 mM solution in THF), copper (I) iodide (0.00072 mmol, 0.144 mL of 5.0 mM solution in CH₃CN) and THF (0.4 mL) were combined in a 2-mL reaction vial to afford a total of 0.9 mL (THF/CH₃CN, 5:1). The sealed vial with the reaction mixture was heated at 90° C. for 25 min in a microwave reactor (Discover 2.0, CEM). The crude reaction mixture was concentrated and the crude solid obtained was purified by column chromatography (silica, 1:4 then 1:2 EtOAc/n-hexanes) to afford a green solid (1.5 mg, 18%). ¹H NMR (CDCl₃, 600 MHz) δ, ppm: −2.03 (bs, 1H), −1.78 (bs, 1H), −1.71 (bs, 1H), −1.51 (bs, 1H), 1.870 (s, 6H), 1.874 (s, 6H), 1.92 (s, 6H), 1.95 (s, 6H), 4.33 (s, 3H), 4.35 (s, 3H), 4.39 (m, 6H), 4.44 (s, 2H), 6.11 (s, 2H), 8.48 (s, 1H), 8.50 (s, 1H), 8.57 (s, 1H), 8.63 (s, 1H), 8.66 (d, J=2.5 Hz, 1H), 8.74 (d, J=2.5 Hz, 1H), 8.88 (d, J=2.0 Hz, 1H), 8.94 (s, 1H), 8.96 (s, 1H), 8.98 (d, J=1.9 Hz, 1H), 9.59 (s, 1H); ¹³C NMR (CDCl₃, 150 MHz) δ, ppm: 14.3, 22.9, 23.5, 24.9, 29.6, 29.9, 31.0, 31.1, 31.2, 31.2, 32.1, 36.9, 42.1, 45.6, 45.6, 46.0, 46.2, 47.5, 48.1, 51.4, 52.2, 64.6, 64.9, 83.4, 87.1, 96.5, 96.5, 96.8, 97.4, 98.1, 98.5, 104.9, 107.2, 113.4, 121.9, 122.6, 124.0, 125.1, 125.4, 125.8, 126.6, 127.7, 134.0, 134.5, 135.3, 135.5, 135.6, 135.8, 136.2, 137.9, 144.8, 154.2, 156.5, 160.8, 162.0, 169.5, 169.6, 170.4, 171.4; λ_{abs} (toluene) 365, 374, 514, 740 nm. λₑₘ (toluene)=742 nm (λₑₓₑ=514 nm). R_{f} (1:3 ethyl acetate:hexanes) 0.40.

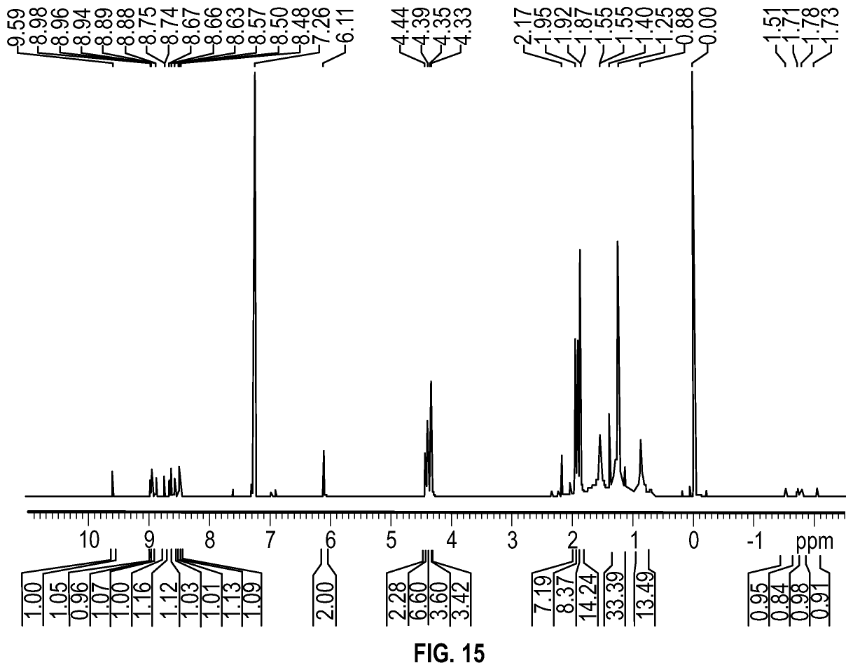

FIG. 15 shows the ¹H NMR spectrum of bis-Br-BC (CDCl₃, 600 MHz).

bis-BC

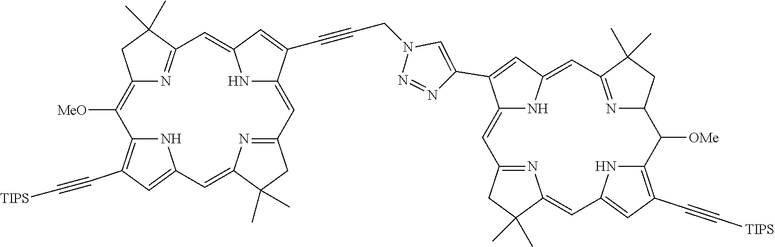

3-(triisopropylsilyl)ethynyl-5-methoxy-13-(3-(triisopropylsilyl)ethynyl)-5-methoxy-8,8,18,18-tetramethyl-bacteriochlorin)-(1-H-1,2,3-triazol-1′-yl)propynyl)-(8,8,18,18-tetramethyl-bacteriochlorin (bis-BC). In a 5-mL round bottom flask, a solution of bis-Br-BC (1.5 mg, 0.0014 mmol) in degassed DMF/Et₃N (0.7 mL, 2:1) was treated with (triisopropylsilyl)acetylene (3.0 μL, 0. mmol), and tetrakis(triphenylphosphine)palladium (0.7 mg, 0.00056 mmol). The reaction mixture was stirred in the sealed flask 5 h at 80° C. Upon reaction completion, the reaction mixture was diluted with ethyl acetate (30 mL), washed with water (30 mL) and brine (30 mL), dried (anhydrous Na₂SO₄) and concentrated to solid. The crude was purified by column chromatography (silica, 1:3 ethyl acetate/n-hexanes) to afford pink solid (1.0 mg, 56%). λ_{abs} (toluene) 359, 379, 521, 533, 741, 766 nm. λₑₘ 769 nm (λ_{exc}=520 nm, toluene). R_{f} (1:3 ethyl acetate:hexanes) 0.50.

### BC-L

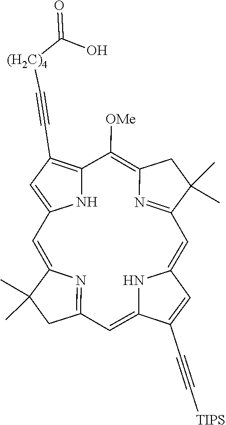

3-(6-carboxyhex-1-ynyl)-5-methoxy-8,8,18,18-tetramethyl-13-(triisopropylsilyl)ethynyl-bacteriochlorin (BC-L). A solution of 3-bromo-13-ethynyl-5-methoxy-8,8,18,18-tetramethyl-bacteriochlorin 3 (9.0 mg, 0.014 mmol), hept-6-ynoic acid (2.5 mg, 0.020 mmol), and triethylamine (1.0 mL) in N,N-dimethylformamide (2.0 mL) in an oven-dried 25-mL Schlenk flask was degassed via three freeze-pump-thaw cycles. Bis(triphenylphosphine)palladium dichloride (1.0 mg, 0.0014 mmol) was added, and the reaction mixture was stirred 80° C. overnight under argon. The reaction mixture was concentrated. The crude was purified by column chromatography (silica, ethyl acetate/hexanes 1:2) to afford a red solid (4.4 mg, 46%). ¹H NMR (CDCl₃, 600 MHz) δ, ppm: −1.96 (bs, 1H), −1.72 (bs, 1H), 1.35-1.36 (m, 21H), 1.916 (s, 6H), 1.919 (s, 6H), 1.95-2.01 (m, 2H), 2.05-2.11 (m, 2H), 2.59 (t, J=7.2 Hz, 2H), 2.86 (t, J=2.86 Hz, 2H), 4.38 (s, 2H), 4.399 (s, 2H), 4.405 (s, 2H), 8.48 (s, 1H), 8.50 (s, 1H), 8.67 (d, J=2.1 Hz, 1H), 8.73 (d, J=1.9 Hz, 1H), 8.86 (s, 1H).

### BC-L-NHS

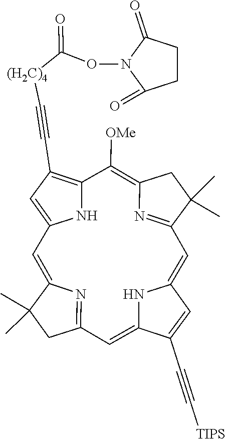

3-(6-(N-succinimidooxy)hex-1-ynyl)-5-methoxy-8,8,18,18-tetramethyl-13-(triisopropylsilyl)ethynyl-bacteriochlorin (BC-L-NHS). A solution of 3-(6-carboxyhex-1-ynyl)-5-methoxy-8,8,18,18-tetramethyl-13-(triisopropylsilyl)ethynyl-bacteriochlorin (BC-L) (4.4 mg, 0.0062 mmol) in anhydrous dichloromethane (0.62 mL) was treated with N-hydroxysuccinimide (1.57 mg, 0.0136 mmol), and 1-ethyl-3-(3-dimethylaminopropyl) carbodiimide hydrochloride (2.12 mg, 0.0136 mmol). The reaction mixture was stirred overnight at room temperature under argon. The reaction mixture was diluted with dichloromethane, washed with water (3×10 mL), and brine (3×10 mL), dried (anhydrous Na₂SO₄), and concentrated to green solid. The crude was purified by column chromatography (silica, EtOAc/n-hexanes, 1:2) to afford a green solid (3.1 mg, 62%). ¹H NMR (CDCl₃, 600 MHz) δ, ppm: −1.96 (bs, 1H), −1.72 (bs, 1H), 1.34-1.39 (m, 21H), 1.921 (s, 6H), 1.925 (s, 6H), 2.00-2.06 (m, 2H), 2.16-2.22 (m, 2H), 2.83-2.90 (m, 8H), 4.38 (s, 2H), 4.40 (s, 5H), 8.49 (s, 1H), 8.51 (s, 1H), 8.68 (d, J=2.2 Hz, 1H), 8.74 (d, J=1.9 Hz, 1H), 8.86 (s, 1H); ¹³C NMR (CDCl₃, 150 MHz) δ, ppm: 11.9, 19.2, 20.2, 24.4, 25.9, 28.4, 29.9, 31.0, 31.1, 31.2, 15.7, 45.8, 47.9, 51.9, 64.7, 96.5, 97.0, 97.6, 125.4, 168.7, 169.3, 170.2.

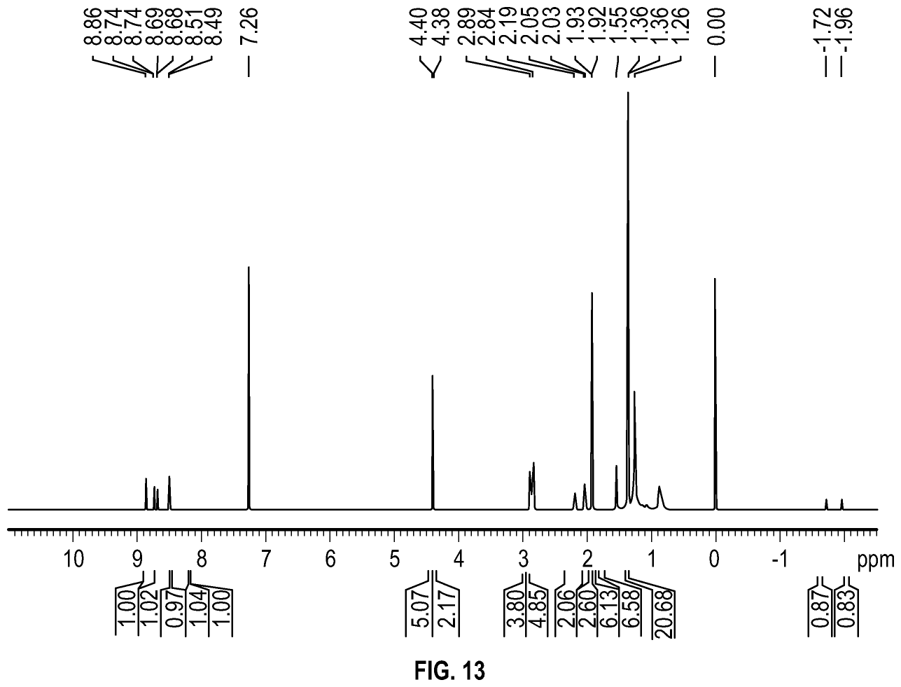

FIG. 13 shows the ¹H NMR spectrum of BC-L-NHS (CDCl₃, 600 MHz).

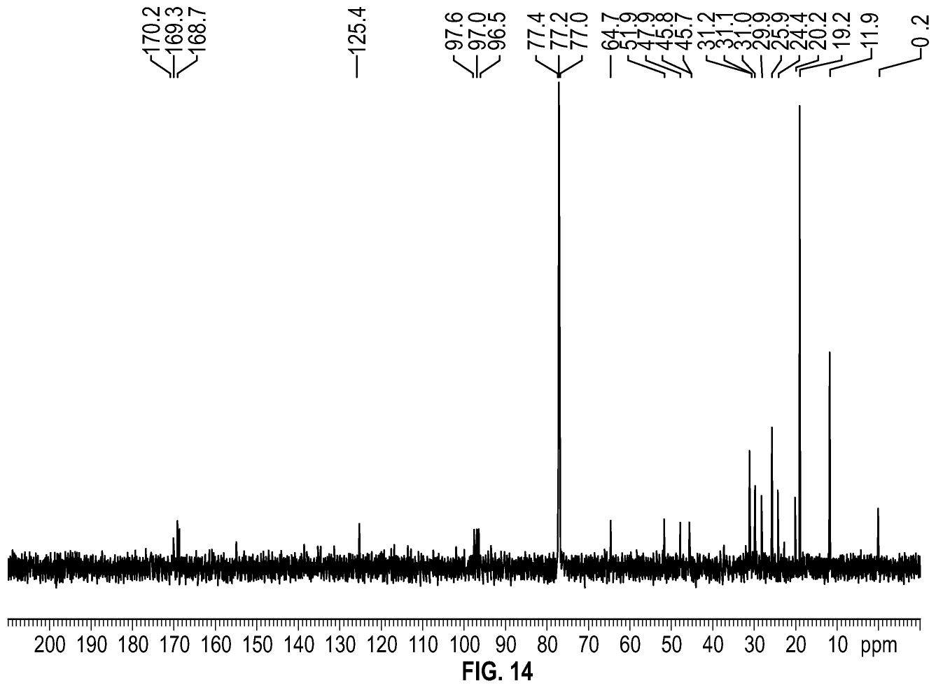

FIG. 14 shows the ¹³C NMR spectrum of BC-L-NHS (CDCl₃, 150 MHz).

### BC-DNA

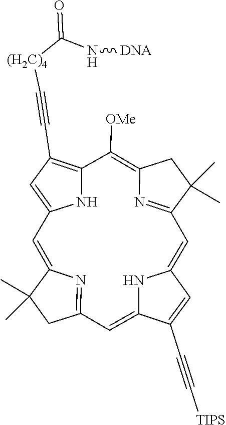

BC-DNA. The Oligomer (5′-ATA TAA TCG CTC G[NH₂—C6-T]C ATA TTA TGA CTG-3′) internally functionalized with amino thymidine sequence modifier [NH₂—C6-T] was conjugated with BC-L-NHS bacteriochlorin and purified via dual high-performance liquid chromatography at Integrated DNA Technologies to afford BC-DNA (0.5 nmoles). HRMS (MS-IALTQ) Calcd 9089.63, found 9090.20.

### 3-(3-(tert-butoxycarbonylamino)propynyl)-5-methoxy-8,8,18,18-tetramethyl-13-(triisopropylsilyl)ethynyl-bacteriochlorin

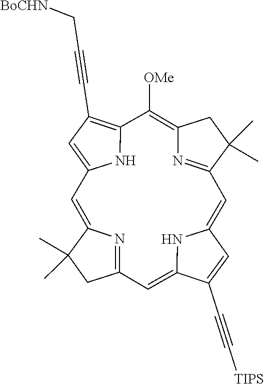

3-(3-(tert-butoxycarbonylamino)propynyl)-5-methoxy-8,8,18,18-tetramethyl-13-(triisopropylsilyl)ethynyl-bacteriochlorin (see e.g., position 408). A solution of 3-bromo-13-ethynyl-5-methoxy-8,8,18,18-tetramethyl-bacteriochlorin, see e.g., position 402 (21.2 mg, 0.032 mmol), and N-(tert-butoxycarbonyl)propargylamine in DMF/Et₃N (9.6 mL, 2:1) was degassed vis three freeze-thaw cycles in 25-mL Schlenk flask. A sample of bis(triphenylphosphine)palladium dichloride (4.5 mg, 0.0064 mmol) was added. The reaction mixture was stirred in the sealed flask for 5 h at 80° C. Upon reaction completion, the reaction mixture was diluted with ethyl acetate (50 mL), washed with water (50 mL) and brine (50 mL), dried (anhydrous Na₂SO₄) and concentrated to green solid. The crude was purified by column chromatography (alumina, 1:2:4 then 1:1:2 ethyl acetate/toluene/n-hexanes) to afford green solid (12.1 mg, 51%). 1H NMR (CDCl3, 600 MHz) δ, ppm: −1.79 (bs, 1H), −1.61 (bs, 1H), 1.34-1.38 (m, 21H), 1.55 (s, 9H), 1.920 (s, 6H), 1.924 (s, 6H), 4.38 (s, 2H), 4.39 (s, 2H), 4.40 (s, 3H), 4.56 (d, J=4.2 Hz, 2H), 5.09 (bs, 1H), 8.48 (s, 1H), 8.49 (s, 1H), 8.66 (d, J=2.1 Hz, 1H), 8.74 (d, J=1.9 Hz, 1H), 8.84 (s, 1H)); 13C NMR (CDCl3, 150 MHz) δ, ppm: 11.8, 19.1, 28.7, 29.9, 31.0, 31.3, 32.3, 45.6, 45.9, 47.5, 52.0, 64.7, 80.2, 80.8, 89.4, 96.5, 97.3, 97.5, 98.9, 101.7, 111.6, 117.7, 124.7, 125.5, 126.0, 128.4, 129.2, 131.3, 134.2, 135.6, 136.1, 139.0, 154.5, 155.8, 161.6, 169.8, 170.7.

### BC1

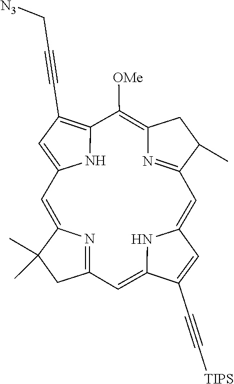

3-(3-azido)propynyl-5-methoxy-13-(triisopropylsilyl)ethynyl-8,8,18,18-tetramethyl-bacteriochlorin (BC1). A solution of 3-(3-(tert-butoxycarbonylamino)propynyl)-5-methoxy-8,8,18,18-tetramethyl-13-(triisopropylsilyl) ethynyl-bacteriochlorin (408) (12.1 mg, 0.016 mmol) in dichloromethane (2.2 mL) in a 5 mL round bottom was treated with trifluoroacetic acid (0.55 mL, 7.2 mmol) all at once, and the solution at 0° C. under argon. The reaction mixture was stirred for 1 h at 0° C. under argon. The reaction mixture was washed with 0.1 M aqueous potassium carbonate (2×40 mL), water (40 mL), and brine (40 mL); dried (anhydrous Na₂SO₄), and concentrated to afford green solid that was used in the next step without purification. ¹H NMR (CDCl₃, 600 MHz) δ, ppm: −1.84 (bs, 1H), −1.64 (bs, 1H), 1.34-1.39 (m, 21H), 1.91 (s, 2H), 1.92 (s, 6H), 1.93 (s, 6H), 4.07 (bs, 2H), 4.39 (s, 2H), 4.40 (s, 2H), 4.43 (s, 3H), 8.49 (s, 1H), 8.50 (s, 1H), 8.68 (d, J=1.7 Hz, 1H), 8.75 (d, J=1.8 Hz, 1H), 8.86 (s, 1H)); ¹³C NMR (CDCl₃, 150 MHz) δ, ppm: 11.8, 19.1, 29.9, 31.0, 31.3, 45.7, 45.8, 47.6, 51.9, 64.7, 96.5, 97.2, 97.5, 98.8, 101.8, 112.4, 117.5, 124.8, 125.8, 131.2, 134.4, 135.5, 135.9, 138.9, 154.6, 161.4, 170.0, 170.6.

A solution of 3-(3-aminopropyl)-5-methoxy-13-(3-amino)propynyl-8,8,18,18-tetramethyl-bacteriochlorin (crude from previous step), triethylamine (0.014 mL, 0.103 mmol), zinc acetate (10.7 mg, 0.055 mmol), and imidazole-1-sulfonyl azide tetrafluoroborate (4.7 mg, 0.017 mmol) in 3:2 dichloromethane/methanol (4.1 mL) was stirred in a 10 mL round bottom flask under argon at room temperature for 2 h. The reaction mixture was washed with water (30 mL), dried (anhydrous Na₂SO₄) and concentrated. The crude was purified by column chromatography (silica, ethyl acetate/n-hexanes 1:4) to afford green solid (3.3 mg, 36%). ¹H NMR (CDCl₃, 600 MHz) δ, ppm: −1.64 (s, 1H), −1.50 (s, 1H), 1.34-1.38 (m, 21H), 1.92 (s, 12H), 4.37 (s, 2H), 4.38 (s, 2H), 4.41 (s, 3H), 4.55 (s, 2H), 8.46 (s, 1H), 8.47 (s, 1H), 8.70 (d, J=2.2 Hz, 1H), 8.73 (d, J=1.8 Hz, 1H), 8.82 (s, 1H)); ¹³C NMR (CDCl₃, 150 MHz) δ, ppm: 11.8, 19.1, 24.9, 29.9, 30.9, 31.3, 36.8, 41.8, 45.5, 46.0, 47.4, 52.1, 64.6, 84.9, 85.1, 96.6, 97.4, 97.6, 99.2, 101.5, 110.3, 118.2, 124.8, 126.4, 131.2, 133.9, 135.6, 136.6, 139.3, 154.2, 162.0, 169.7, 171.3.

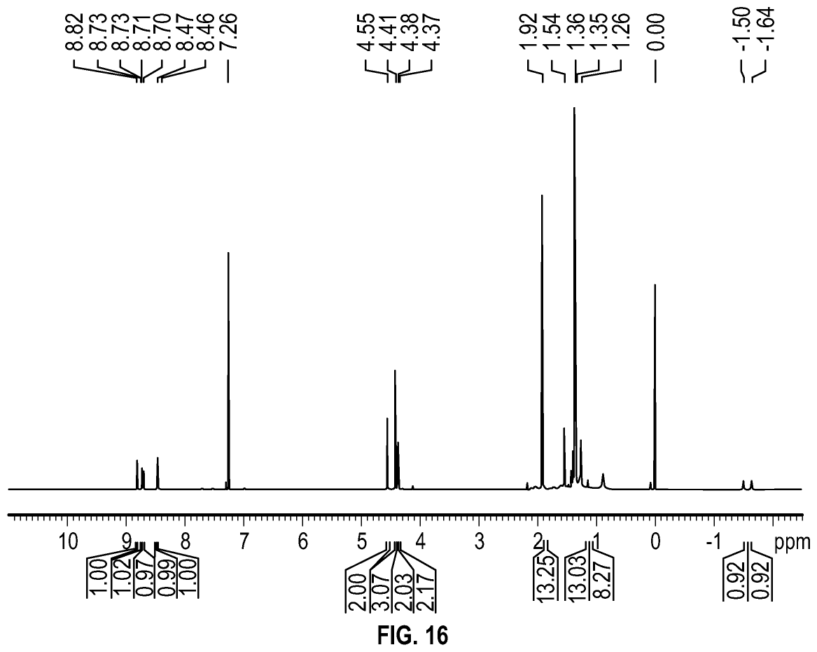

FIG. 16 shows the ¹H NMR spectrum of BC-1 (CDCl₃, 600 MHz).

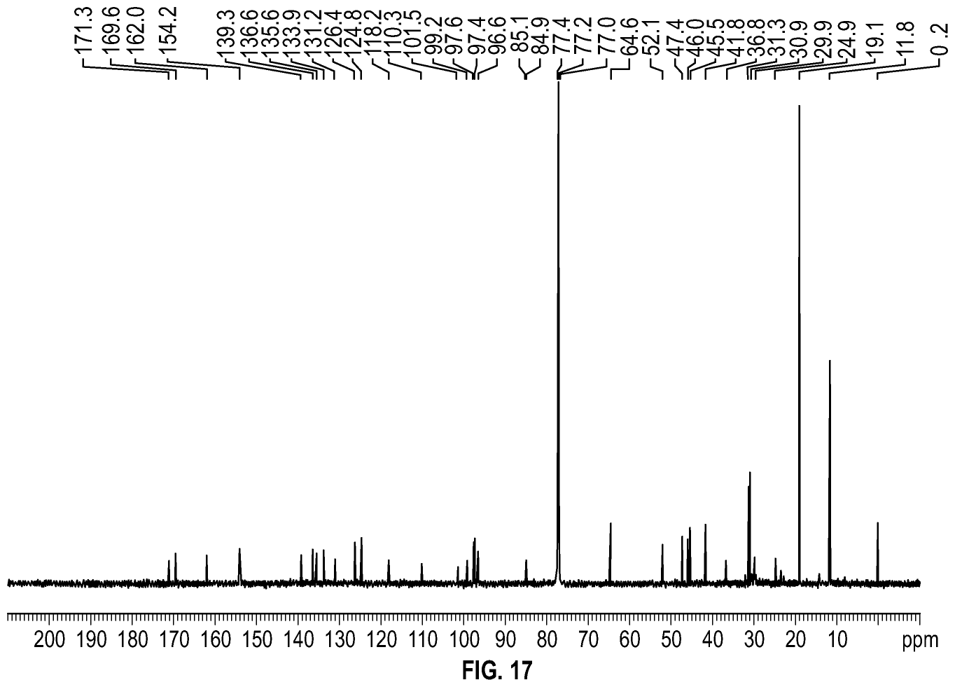

FIG. 17 shows the ¹³C NMR spectrum of BC-1 (CDCl₃, 150 MHz).

alkyne-BC

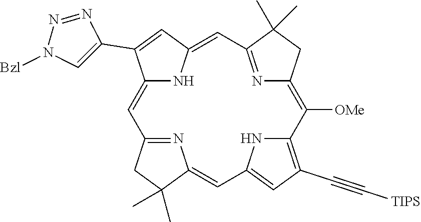

3-3-(triisopropylsilyl)ethynyl-5-methoxy-13-(4′-benzyl-(1-H-1,2,3-triazol-1′-yl))-(8,8,18,18-tetramethyl-bacteriochlorin (triazole-BC). A solution of 3-bromo-13-ethynyl-5-methoxy-8,8,18,18-tetramethyl-bacteriochlorin (alkyne-BC) (6.0 mg, 0.0119 mmol) and benzyl azide (4.5 μL, 0.0358 mmol) were dissolved in THF (0.34 mL) in a 5 mL round-bottom flask. A solution of diisopropylethylamine in THF (600 □L, 10 mM, 0.0060 mmol) was added, followed by a solution of copper (I) iodide in acetonitrile (478 □L, 5.0 mM, 0.00024 mmol). The flask was sealed, and heated at 60° C. for 4 hours while stirring. The reaction mixture was cooled to room temperature and concentrated to dryness. The crude was purified by column chromatography (silica, 1:3 EtOAc/hexanes) to afford green solid of 3-bromo-5-methoxy-13-(4′-benzyl-(1-H-1,2,3-triazol-1′-yl)-(8,8,18,18-tetramethyl-bacteriochlorin (3.8 mg, 50%). 1H NMR (CDCl3, 600 MHz) δ, ppm: −1.75 (bs, 1H), −1.53 (bs, 1H), 1.91 (s, 6H), 1.93 (s, 6H), 4.33 (s, 3H), 1.39 (s, 2H), 4.40 (s, 2H), 5.82 (s, 2H), 7.44 (m, 1H), 7.50 (m, 4H), 8.25 (s, 1H), 8.48 (s, 1H), 8.53 (s, 1H), 8.66 (d, J=2.5 Hz, 1H), 8.83 (d, J=2.0 Hz, 1H), 9.49 (s, 1H); 13C NMR (CDCl3, 150 MHz) δ, ppm: 31.0, 31.2, 45.6, 46.2, 47.5, 52.3, 54.7, 64.6, 96.9, 97.2, 98.7, 104.7, 121.8, 122.3, 123.9, 125.2, 126.5, 128.5, 129.2, 129.5, 133.9, 135.0, 125.5, 135.6, 136.2, 144.7, 154.1, 162.1, 169.4, 170.4; λabs 362, 374, 511, 730 nm (toluene); λem 733 nm (λexc 511 nm, toluene). Rf 0.24 (1:3 ethyl acetate:hexanes).

In a 5-mL round bottom flask, a solution of of 3-bromo-5-methoxy-13-(4′-benzyl-(1-H-1,2,3-triazol-1′-yl)-(8,8,18,18-tetramethyl-bacteriochlorin (1.9 mg, 0.0029 mmol) in degassed DMF/Et3N (5.3 mL, 2;1) was treated with (triisopropylsilyl)acetylene (3.0 μL, 0.0145 mmol) and tetrakis(triphenylphosphine)palladium (0.9 mg, 0.00058 mmol). The reaction mixture was stirred in the sealed vial for 5 h at 80° C. under argon. The reaction mixture was diluted with ethyl acetate (30 mL), washed with water (30 mL) and brine (30 mL), dried (anhydrous Na2SO4), and concentrated. The residue was purified by column chromatography (silica, 1:1 EtOAc/hexanes 1:3) to afford a green solid (1.9 mg, 89%). 1H NMR (CDCl3, 600 MHz) δ −1.69 (bs, 1H), −1.43 (bs, 1H), 1.33-1.34 (m, 21H), 1.90 (s, 6H), 1.92 (s, 6H), 4.39 (s, 4H), 4.41 (s, 3H), 5.82 (s, 2H), 7.44 (m, 1H), 7.47-7.51 (m, 4H), 8.24 (s, 1H), 8.47 (s, 1H), 8.50 (s, 1H), 8.75 (d, J=2.2 Hz, 1H), 8.80 (d, J=1.8 Hz, 1H).

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| CC | covalent coupler |
|---|---|
| D | dye |
| 100 | long polymer aggregate created by dye coupling mediated by a |
| | nucleic acid template |
| 102 | seed dye attached to dNA |
| 104 | dye monomer |
| 106 | dimer |
| 110 | long polymer aggregate created by dye coupling |
| 112 | addition of a catalyst (e.g. light exposure or addition of an |
| | enzyme) |
| 114 | chain |
| 116 | introduction of DNA |
| 118 | tether |
| 120 | long polymer aggregate created by dye coupling in a chain |
| | followed by tethering into DNA |
| 122 | first DNA strand |
| 124 | second DNA strand |
| 200 | superdye (i.e. a dye that can include a non-conjugating bridge) |
| 202 | dye monomer |
| 204 | first covalent coupler |
| 206 | second covalent coupler |
| 208 | protecting group |
| 210 | non-conjugating bridge (e.g., covalent, non-conjugating bridge) |
| 300 | synthetic routes to install azide and alkyne |
| 301 | first position |
| 302 | second position |
| 303 | third position |
| 304 | fourth position |
| 305 | fifth position |
| 306 | sixth position |
| 400 | routes toward monomers and aggregate subunits equipped with |
| | azide/alkyne functional groups |
| 402 | second [seventh] position |
| 408 | eighth position |
| 409 | ninth position |
| 500 | DNA-mediated dye polymer synthesis |
| 501 | first step |
| 502 | second step |
| 503 | third step |
| 504 | fourth step |
| 505 | fifth step |
| 506 | sixth step |
| 600 | best scoring structure of the DNA-bacteriochlorin with propargyl |
| | azide group located in the minor groove and alkyne group pointed |
| | outward |
| 602 | bacteriochlorin |
| 604 | click chemistry |
| 606 | DFT-optimized structure of the bacteriochlorin dimer fragment |
| 702 | superdye dyad |
| 703 | superdye triad |
| 704 | superdye tetrad |
| 705 | superdye pentad |
| 800 | excitonic quantum-controlled basis change gate |
| 802 | phase shifting element |
| 804A | first basis change gate |
| 804B | second basis change gate |
| 806 | transmission line |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

When referring to numerical values, “approximately”, in the absence of context indicating otherwise, means +/−10%.

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

An “aggregate”, as used herein, exhibits “exciton delocalization”.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. An aggregate whose length are controllable, comprising:
a chemical reaction of dye monomers, dyes, or aggregate sub-units that create a polymer chain of polymer dye aggregates, and
a covalent, non-conjugating bridge forming upon the coupling and connecting neighboring dye monomers, dyes, or aggregate sub-units in the polymer chain.

2. The aggregate of claim 1, wherein dyes are linked into the polymer chain by dye coupling mediated by a DNA template, a protein template or a metal-organic framework (MOF) template.

3. The aggregate of claim 1, wherein dyes are linked into the polymer chain by dye coupling, followed by a chain tethering to a DNA template.

4. The aggregate of claim 1, wherein the dyes are linked into the polymer chain by tethering the dyes as monomers to a DNA template followed by the chemical reaction.

5. The aggregate of claim 3, wherein the dye chain or dye monomers are:
tethered to the same single strand;
tethered to opposing strands within one DNA duplex; or
tethered to different DNA duplexes.

6. The aggregate of claim 1, wherein the chemical reaction is a click-chemistry coupling comprising dyes coupled via a copper(I)-catalyzed azide-alkyne cycloaddition (CuAAC) reaction between an azide and alkyne to form a 1,2,3-triazole ring linking the dyes, wherein a copper catalyst contains copper(I)-stabilizing ligands (e.g., tris(benzyltriazolylmethyl)amine, TBTA3) to prevent DNA damage (strand breaks) during CuAAC.

7. The aggregate of claim 6, wherein the dyes include couplings (1) in the presence of DNA; (2) prior to the DNA templating, or (3) after the monomers are tethered to the DNA, and further wherein the dyes are modified with the azide-containing group and with a terminal alkyne group.

8. The aggregate of claim 7, wherein the terminal alkyne group is protected with a trialkyl silyl moiety selected from the group consisting of triisopropylsilylalkyne (TIPS-alkyne), trimethylsilyl alkyne (TMS-alkyne), t-butyldimethylsilylalkyne (TBDMS), thexyldimethylsilylalkyne (TDS-alkyne), benzyl dimethylsilyl alkyne (BDMS-alkyne), biphenyl dimethylsilyl alkyne (BDMS-alkyne), biphenyldiisopropylsilylalkyne (BDIPS-alkyne) and tris(biphenyl-4-yl)silyl (TBPS-alkyne), said trialkyl silyl moiety being in the form of a trialkyl silyl acetylene.

9. The aggregate of claim 1, wherein a dye family chosen for dye coupling into the polymer chain is a click-chemistry coupling and comprises a synthetic access to install coupling functional groups.

10. The aggregate of claim 9, wherein at least one coupling group is protected to inhibit chemical activity of the at least one coupling group and to prevent undesired self-coupling, and further wherein the functional groups couple under DNA-compatible conditions using a protection-deprotection coupling strategy.

11. The aggregate of claim 1, wherein and the bridge is short (3-6 Å) to ensure excitonic coupling between the dyes, and further wherein the non-conjugated dye polymer is synthesized via successive coupling reactions of the dye monomers on DNA.

12. The aggregate of claim 1, wherein synthetic access to dye monomers and/or dyes suitable for the chemical reaction comprise synthetic routes to independently install alkyne and azide groups.

13. The aggregate of claim 12, wherein:
the synthetic routes start with a 3,13-dibromobacteriochlorin building block; and
a terminal alkyne is protected with a triisopropylacetelyne group.

14. The aggregate of claim 12, wherein the alkyne and azide groups are installed in the dye simultaneously and a covalent linker is attached to the dye.

15. The aggregate of claim 1, wherein each dye is equipped with two coupling groups on opposite sides of a dye's transition dipole moment.

16. The aggregate of claim 1, wherein the dyes are covalently linked to each other and align in a head-to-tail fashion.

17. The aggregate of claim 1, wherein a first dye is covalently attached to an oligo strand termini (3′ or 5′ end) or internally via a linker.

18. The aggregate of claim 1, wherein dye polymer growth is carried out on (i) dsDNA or (ii) with a single-stranded DNA, RNA, or oligonucleotide in solution or attached to a solid phase.

19. The aggregate of claim 1, further comprising a nucleic acid single strand hybridized with a complementary strand.

20. The aggregate of claim 1, further comprising a DNA minor groove along which the polymer chain is routed.
